(19) United States
(12) Patent Application Publication
(10) Pub. No.: US 2002/0168771 A1
(43) Pub. Date: Nov. 14, 2002

(54) Vectors having replication, immunogenicity and/or pathogenicity under stress promoter regulation and use thereof

(76) Inventor: Gary Eric Gamerman, Vienna, VA (US)

Correspondence Address: Gary Eric Gamerman, 2158 Bonaventure Drive, Vienna, VA, 22181, US

(21) Appl. No.: 09/850,270

(22) Filed: May 8, 2001

(51) Int. Cl. C12N 15/867; C12N 15/861; C12N 7/00; C12N 15/74

(52) U.S. Cl. 435/456; 435/235.1; 435/320.1

(57) ABSTRACT

The present invention relates to modified vectors, e.g. plasmids, viruses or microbia sucsh as yeast or bacteria, wherein the replication, immunogenicity and/or pathogenicity is placed under the control of at least one stress gene regulating element. In preferred embodiments, these modified vectors are used for gene therapy, in vaccines, or for functional genomic screening.

### FIELD OF THE INVENTION

The present invention relates to vectors, e.g., bacterial, yeast, viral or plasmid vectors, wherein the replication, immunogenicity and/or pathogenicity of such vectors is modulated by a stress gene regulatory sequence. The invention also relates to the use of such vectors for treatment and/or prophylaxis of disease, and for diagnostic or screening assays, e.g., genomics research.

### BACKGROUND OF THE INVENTION

It is well known that when placed under permissive conditions, microbial organisms such as bacteria, fungi, protozoans, and viruses as well as autonomously replicating genetic elements such as plasmids avidly replicate, consume, metabolize and express a wide variety of proteins and biological molecules. This replication can be beneficial or pathogenic. For example, in humans and other hosts, uncontrolled replication of bacteria, viruses or other microbia is responsible for their pathogenicity and other harmful effects. Indeed, unfettered replication of viruses in host cells typically results in cell death.

It is also well known that the replication capacity of microbia can be harnessed and channeled for beneficial purposes under appropriate conditions, i.e., by controlling the cellular environment of where replication takes place and/or by replicating or limiting the replication capacity of such microbia.

For example, in the field of therapeutics, it is well known to produce attenuated microbia or genomic components thereof (e.g., autonomously replicating plasmids) that have an altered (more limited) replication capacity or which express pathogenic traits less effectively (e.g., avirulent or attenuated bacterial mutants). This strategy is useful for preparing attenuated microbia for use in vaccines that immunize the host against the wild-type organism both which do not themselves cause the disease. Examples thereof include the use of attenuated microbia or viruses to immunize against polio, chicken pox, salmonella, and pertussis, or the use of such attenuated microbia to boost general or site specific immunity. The latter strategy has been used especially in the treatment of bladder cancer (with an attenuated BCG). Also, the use of attenuated microbia or genomic components in gene therapy is well known. For example, the use of attenuated adenoviral, adeno-associated viruses, herpes viruses and retroviruses for gene therapy is known.

It has also been recognized by those skilled in the art that it would be desirable to produce vectors wherein the replication and/or expression of pathogenic characteristics is subjected to efficient regulation, i.e., such that it is only expressed at specific times, locations and/or conditions, most preferably reversibly. Such vectors are desirable relative to an otherwise “normal” or “wild-type” pathogen, because the vector replicates only within certain parts of the body and/or only at specific time and conditions. These vectors are potentially useful in treating tumor or immunological conditions, or for eliciting a potent immune responses, e.g., against a pathogen. These approaches have been used in the content of modified adenoviruses, retroviruses and herpes viruses in particular.

Examples thereof the mutant E1b adenoviruses reported by Onyx Pharmaceuticals, Inc., U.S. Pat. No. 6,080,578, issued on Jun. 27, 2000. This patent describes the treatment of neoplastic diseases using mutant viruses lacking p53 or RB gene which are administered to a patient having cancer characterized by cells lacking p53 and/or RB function. These viruses preferentially replicate in neoplastic but not normal cells resulting in selective killing of neoplastic cells either directly or by expression of a cytotoxic gene.

Attenuated adenoviruses are also disclosed in U.S. Pat. Nos. 5,846,945, 5,677,178 and 5,801,029 assigned to Onyx Pharmaceutical Inc. The use of such vectors for killing dividing cancer cells and endothelial cells which proliferate but which do not kill quiescent (non-dividing) normal cells also reported in WO 024408A1. Mutant adenoviruses defective in the E3 region, as well as E1b or E1a region are further disclosed in WO 995583/A2, published Nov. 4, 1999.

The construction of chimeric viruses, the replication of which is regulated by a transactivation signal produced by diseased cells is also known. (See WO 9704805/A1, by Onyx Pharmaceuticals, published Feb. 13, 1997).

Targeted gene therapy using a recombinant virus, particularly adenovirus, wherein replication is placed under the control of a heterologous tissue-specific transcriptional regulatory sequence is also disclosed in U.S. Pat. No. 5,998,205 by Hallenbeck et al., assigned to Genetic Therapy, Inc.

The construction of a viral vector that have been engineered to contain a synthetic promoter that controls at least on essential gene that is induced by a specific protein not normally produced by the viral infected cells is also described in U.S. Pat. No. 6,110,744, by Fang et al., and assigned to the University of Texas. Still further, the construction of adenoviral vectors that selectively replicate in breast cancer cells is reported in U.S. Pat. No. 6,096,718 by Weitzman et al., assigned to Gene Targeting Corp.

The construction of replication-defective adenoviruses that comprise deletions in the E1a, E1b and E3 gene for use in treating lung disorders has also been reported in U.S. Pat. No. 6,013,638, issued Jan. 11, 2000 to Crystal et al., and jointly-assigned to the U.S. Government, National Institutes of Health and Transgene, S. A.

Still further, the construction of replication-defective adenoviral vectors specific for cells that comprise a specific transcriptional response element (PB-TRE), particularly androgen receptor expressing cells is described in U.S. Pat. No. 6,197,293, issued Mar. 6, 2001 to Henderson et al., and assigned to Calydon, Inc. Also, the construction of adenoviral vectors wherein replication is under control of prostate cell specific transcription response element is reported in U.S. Pat. No. 5,698,443, issued Dec. 16, 1997 to Henderson et al., also assigned to Calydon, Inc.

Attenuated replication-defective HIV vectors and the use thereof in gene therapy and vaccines is also disclosed in U.S. Pat. No. 6,207,455 issued Mar. 22, 2001 to Lung-Ji Chang.

Replication defective retroviruses for infecting human cells are also reported in U.S. Pat. No. 6,133,029 issued Oct. 17, 2000 to Gruber et al., and assigned to Chiron Corporation. The above-identified references disclosing replication modified viruses are exemplary of the state of the art.

However, notwithstanding which has been previously reported, it would be extremely useful if improved vectors wherein the replication, immunogenicity and/or pathogenicity is regulatable under tight temporal, spatial or other conditional controls were developed.

The use of stress inducible promoters to direct the expression of heterologous DNAs has also been reported. (See e.g., Dreano et al., Gene 49:1-8 (1986); WO 89/59822 by Dreano et al., assigned to Intracel Corporation; WO 90/01543 by Dreano et al., assigned to Intracel Corporation; WO 87/05935 by Bromley et al., assigned to Batelle Memorial Institute at Columbia and Ohio Geneva Research Center; WO 87/00861, by Bromley et al., assigned to Batelle Memorial Institute; U.S. Pat. No. 5,614,381 issued Mar. 25, 1997 to Bromley et al., assigned to Rothwell Properties Limited; U.S. Pat. No. 5,646,010 issued Jul. 8, 1997, also assigned to Rothwell Properties Inc.; WO 89/00603 published Jan. 26, 1989, by Bromley et al., assigned to Intracel Corporation; and WO 88/02778, published Apr. 21, 1988 by Bromley et al., and assigned to Batelle Memorial Institute. Also, the use of this technology for gene therapy has been reported (See e.g., WO 99/42137 published Aug. 26, 1999 by Bromley et al.).

Additionally, the use of stress inducible promoters to regulate expression of therapeutic genes expressed in selected cells by use of local controlled heating has been reported in WO 98/06817 by Mooneh Chrit., which application is assigned to the U.S. Government, National Institutes of Health.

Still further, the use of the stress inducible promotes in vitro and in vivo in transgenic animals that screen biological, physical and chemical toxic agents has been reported in WO 99/11772 published Mar. 11, 1991 by Roncucci et al.; Cannon et al., Nature Biotech. 15: 1344-1357 (1997); Fischbach et al., Cell Biol. Toxicol. 9(2): 177-188 (1993); and Fischbach et al., Chapt. 11, “Stress Reporter Gene Assays for In Vitro Toxicity Assessment”.

### OBJECTS OF THE INVENTION

By contrast, it is an object of the invention to utilize stress inducible regulatory elements, e.g., heat shock regulatory elements to regulate the replication, immunogenicity and/or pathogenicity of an autonomously replicating vector which in its wild-form does not replicate and/or exhibit pathogenicity or immunogenicity under heat shock control.

It is a more specific object of the invention to provide vectors, e.g., plasmids, viruses, bacteria, protozoans or fungi wherein the replication, immunogenicity and/or pathogenicity of such vectors is modulated by one or more stress inducible, e.g., heat shock regulatory sequences.

It is an even more specific object of the invention to provide viral vectors, e.g., adenoviral, retroviral (HIV, lentivirus, etc.) poxvirus (e.g., vaccinia virus), herpesvirus and others, wherein the replication, immunogenicity and/or pathogenicity of such viral vector is modulated by placing a vector gene under the control of at least one stress inducible, e.g., heat shock regulatory sequence.

It is another even more specific object of the invention to provide adenoviral vectors wherein the replication, immunogenicity and/or pathogenicity of such viral vector is modulated by placing a gene, e.g. an adenoviral replication gene, under the control of at least on heat shock regulatory sequence.

It is another specific object of the invention to combine such vectors or modify such vectors by the incorporation of one or more therapeutic agents or a gene encoding such as cytokines, toxins, enzymes, apoptosis affecting agents, cell cycle affecting agents, angiogenesis modulators, tumor suppressors, pro-drugs, and others.

It is another object of the invention to administer a vaccine comprising a vector wherein the replication, immunogenicity and/or pathogenicity is modulated by placing at least one vector gene under the control of at least one stress inducible regulatory sequence, e.g., heat shock regulatory element, to treat a subject in need of such administration, e.g., an immuno-compromised subject, or a subject to be immunized against a specific antigen, e.g. a virus, bacterium or fungi.

It is another object of the invention to provide a method of targeted gene therapy using a vector, the replication, immunogenicity and/or pathogenicity of which is regulated by at least one stress inducible regulatory sequence, e.g., heat shock regulatory sequence, and further comprising at least one therapeutic agent or a gene, which vector is administered to a subject, and is induced to replicate, exhibit pathogenicity and/or immunogenicity at a target site, e.g., a tumor or site of an infection for a desired time period.

It is another object of the invention to assay the function of a particular DNA sequence by administering a vector expressing said DNA sequence wherein the replication, immunogenicity and/or pathogenicity of such vector is under the control of at least one stress inducible regulatory sequence, e.g., heat shock promoter regulating sequence; administering said vector to a host, inducing the replication, immunogenicity and/or pathogenicity of said vector at a specific site in said host and evaluating the effect of the expression of said DNA on the host vis-a-vis a control.

It is another object of the invention to selectively ablate cells or tissues at a target site in a human or non-human animal by administering a vector, the replication, immunogenicity and/or pathogenicity of which is under the control of at least one stress inducible regulatory element, e.g., heat shock regulatory element, and optionally further comprising a DNA encoding a cytotoxic, diagnostic or therapeutic agent; and inducing the replication, immunogenicity and/or pathogenicity of the vector at the target site thereby resulting in selection ablation of cells or tissues at such site.

It is another object of the invention to provide pharmaceutical compositions for therapy or prophylaxis that comprise a therapeutically or prophylactically effective amount of at least one vector the replication, immunogenicity and/or pathogenicity is modulated by at least one stress inducible regulatory element, e.g., heat shock regulatory element, and optimally comprising at least one DNA encoding a toxic, diagnostic or therapeutic agent, and a pharmaceutically acceptable carrier.

It is another object of the invention to provide kits for therapy or prophylaxis that comprise:

1 (i) at least one vector the replication, immunogenicity and/or pathogenicity which is regulated by at least one heat shock regulatory element and optimally comprising a DNA encoding a desired polypeptide, e.g., a therapeutic, cytotoxic, diagnostic or cell-affecting agent;

1 (ii) packaging means; and

1 (iii) appropriate instructions for the user.

It is still another object of the invention to provide vaccines for conferring immunity against a target antigen that comprise a vector that expresses the target antigen, wherein replication, immunogenicity and/or pathogenicity of the vector is modulated by at least one heat shock regulatory element.

### DETAILED DESCRIPTION OF THE INVENTION

A. Definitions

Unless otherwise defined, all terms have their ordinary meaning in the relevant art. To facilitate an understanding of the invention, the following terms are defined.

“Vector,” according to the present invention this refers to a replicating genomic containing moiety, e.g., a plasmid or virus or microorganism, bacterium, yeast, fungi or protozoan. Preferably, the vector will comprise a virus.

“Modified Vector,” according to the present invention refers to a vector that has been modified by the introduction of one or more regulatory elements derived from a stress gene, e.g., a heat shock protein gene, and preferably a heat shock promoter such as HSP70 or HSP70B, operably linked to a gene which affects the replication, pathogenicity and/or immunogenicity of said vector thereby causing replication, immunogenicity, and/or pathogenicity to be by said stress gene regulatory sequence.

“Stress Protein,” according to the present invention this refers to proteins that have their expression induced directly or via signal transduction substantially only under conditions of cellular stress, such as infection, exposure to thermal or other radiation, or toxicity. A well known genus of stress promoters are the heat shock proteins. Examples of stress proteins include CAT5, CPH1, C4P2, DDR2, RPR2, HSC82, HSP47, HSP70.1, HSP90, HSP104, HSP12, HSP150, HSP26, HSP42, HSP60, HSP70, HSP70B, HSP106, HSP78, HSP82, KAR2, MDJ1, S1W1, SUD2, SSA1, SSA2, SSA3, SSB1, SSE1, SSB2, SSC1, SSE2, ST1 , T1P1, TPS2, UB14, 4DT1, ubiquitin, crystalline, rapamycin, P-glycoprotein, metallothionein 1, metallothionein 2, metallothionein 1A, metallothionein 1G.

“Heat Shock Proteins” according to the invention, these refer to a family of proteins encoded by genes that are well conserved across eukaryotes in general that are expressed when cells become exposed to elevated temperature, e.g., about 35-37° C. in the case of Drosophila cells, and which are similar or are expressed at substantially diminished levels at ambient temperatures. During heat treatment these polypeptides are synthesized almost exclusively and after 8 hours can represent 10% of the total cellular protein (See McKenzie et al., 72: 1117-1121 (1975); Mirault et al, Cold Spring Harbor Symp. Quant. Biol. 42: 819-827 (1978); Livak et al., Proc. Natl. Acad. Sci., USA 75: 5613-5617 (1978); Schedl et al., Cell 17: 921-929 (1978); Holmgren et al., Cell 18: 1359-1370 (1974); Wadsworth et al., Proc. Natl. Acad. Sci. USA 77: 2134-2137 (1980); Corces et al., Proc. Natl. Acad. Sci. USA 77: 5390-5393 (1980); Voellmy et al., Cell 23:261-270 (1981); Karch and Torok, Nucl. Acids Res. 8:3105-3123 (1980); Ingola and Cravy, Proc. Natl. Acad. Sci. USA 79: 2360-2364 (1982), all of which are incorporated by reference herein).

Examples of heat shock proteins include by way of example, HSP12, HSP26, HSP42, HSP47, HSP70, HSP70B, HSP60, HSP70.1, HSP78, HSP82, HSP90, HSP104, HSP150.

As noted heat shock proteins and genes are conserved across eukaryotes in general, and have been cloned from many species including Drosophila melanogastor, human, mouse, rat, monkey, Xenopus, et al. Preferred heat shock regulatory elements are those derived from the HSP70 and HSP70B genes, e.g. those from Drosphila melanogaster, as these promoters have been well characterized.

“Replication” in the present invention typically refers to the proliferation of the vector, e.g., a virus, plasmid or bacteria under appropriate (stress-induced) conditions.

“Pathogenicity” in the present invention refers to trait that affect the ability of the vector to cause some adverse effect to a host or host cells that is normally exhibited by the wild-type vector, e.g., killing or lysis of infected cells, e.g., facilitated by the expression of a toxic moiety.

“Inhibition of pathogenicity” refers to the cessation or amelioration of biological effects associated with pathogenicity.

“Immunogenicity” in the present invention typically refers to the vector's ability to elicit an immune response, e.g., one provoked by an immunodominant epitope. As discussed infra, in one embodiment of the invention the immunogenicity of a vector may be altered by placing an immunodominant epitope under the control of a stress protein regulatory element.

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. The term nucleic acid is used interchangeably with gene, cDNA, and mRNA encoded by a gene.

The phrase “exogenous” or “heterologous nucleic acid” generally denotes a nucleic acid that has been isolated, cloned and ligated to a nucleic acid with which it is not combined in nature, and/or introduced into and/or expressed in a cell or cellular environment other than the cell or cellular environment in which said nucleic acid or protein may typically be found in nature. The term encompasses both nucleic acids originally obtained from a different organism or cell type than the cell type in which it is expressed, and also nucleic acids that are obtained from the same cell line as the cell line in which it is expressed.

The phrase “a nucleic acid sequence encoding” refers to a nucleic acid which contains sequence information for a structural RNA such as rRNA, a tRNA, or the primary amino acid sequence of a specific protein or peptide, or a binding site for a transacting regulatory agent. This phrase specifically encompasses degenerate codons (i.e., different codons which encode a single amino acid) of the native sequence or sequences which may be introduced to conform with codon preference in a specific host cell.

The term “recombinant” or “engineered” when used with reference to a nucleic acid or a protein generally denotes that the composition or primary sequence of said nucleic acid or protein has been altered from the naturally occurring sequence using experimental manipulations well known to those skilled in the art. It may also denote that a nucleic acid or protein has been isolated and cloned into a vector, or a nucleic acid that has been introduced into or expressed in a cell or cellular environment other than the cell or cellular environment in which said nucleic acid or protein may be found in nature.

The term “recombinant” or “engineered” when used with reference to a cell indicates that the cell replicates or expresses a nucleic acid, or produces a peptide or protein encoded by a nucleic acid, whose origin is exogenous to the cell. Recombinant cells can express nucleic acids that are not found within the native (nonrecombinant) form of the cell. Recombinant cells can also express nucleic acids found in the native form of the cell wherein the nucleic acids are re-introduced into the cell by artificial means.

A cell has been “transformed” by an exogenous nucleic acid when such exogenous nucleic acid has been introduced inside the cell membrane. Exogenous DNA may or may not be integrated (covalently linked) into chromosomal DNA making up the genome of the cell. The exogenous DNA may be maintained on an episomal element, such as a plasmid. In eucaryotic cells, a stably transformed cell is generally one in which the exogenous DNA has become integrated into the chromosome so that it is inherited by daughter cells through chromosome replication, or one which includes stably maintained extrachromosomal plasmids. This stability is demonstrated by the ability of the eucaryotic cell to establish cell lines or clones comprised of a population of daughter cells containing the exogenous DNA.

“Heat-inducible promoter.” A promoter is a nucleic acid sequence associated with a gene that controls the transcription of the gene by interacting with mainly transacting ligands such as polymerases, transcription factors, transcription enhancers and transcription suppressors. Promoters can be either constitutive or inducible. A constitutive promoter promotes the constant transcription of a gene, whereas an inducible promoter's activity fluctuates as determined by the presence (or absence? of a specific inducer. The regulatory elements of an inducible promoter are usually located further upstream of the transcriptional start site than the TATA box. Ideally, an inducible promoter should possess the following properties: a low to nonexistent basal level of expression in the absence of inductive stimulus, a high level of expression in the presence of inductive stimulus, and an induction scheme that does not otherwise alter the physiology of the cell. A heat inducible promoter is one that is activated by exposing cells that contain the promoter to a defined temperature.

A “host cell” is a cell which has been transformed by an exogenous DNA sequence. Unless otherwise specified, the host cell may be a plant or animal cell.

The term “tumor cell” or “cancer cell” or “neoplastic cell” denotes a cell that demonstrates inappropriate, unregulated proliferation. A “human” tumor is comprised of cells that have human chromosomes. Such tumors include those in a human patient, and tumors resulting from the introduction into a nonhuman host animal of a malignant cell line having human chromosomes into a nonhuman host animal.

“Selectively heating” means that only cells having predetermined spatial coordinates in an organism, tissue or cell mass are directly heated by the heat source, whereas cells that are outside of these coordinates, even though adjacent, are not directly heated. Heating of adjacent cells that may occur by normal heat equilibration between the selectively heated cells and the adjacent cells is a consequence of selective heating.

The term “pro-molecule” refers to a substance that is itself not metabolically active as administered, but that is activated either when chemically altered or metabolized by cells capable of altering or metabolizing the pro-molecule, or when combined with one or more other substances to form a complex that is metabolically active. A pro-molecule may, upon chemical alteration or combination with a second substance, become toxic to the host cell. Examples include 5 fluorocytosine (“5FC”), which can be converted to the lethal metabolite 5-fluorouracil (“5FU”); 5-methoxypurine arabinoside; and gancyclovir. Pro-molecules preferably cause no substantially no ill effects to an organism except to cells that are capable of converting the pro-drug to a toxic product (i.e., metabolite or complex). “Pro-drug activating molecule” denotes a molecule such as an enzyme that is capable of metabolizing the non-toxic pro-drug to its toxic metabolite, or a molecule that combines (covalently or non-covalently) with a pro-drug to yield a toxic product.

A “toxic molecule” is a molecule that inhibits cell growth, or inhibits certain metabolic pathways, in some instances killing the cell. See, e.g., WO 93/24136.

The phrase “therapeutic dose” or “therapeutic amount” or “effective amount” means a dosage sufficient to produce a desired result. The desired result can be subjective or objective improvement in the recipient of the dosage, a decrease or an increase in the number of a target population of cells, a decrease in tumor size, a decrease in the rate of growth of cancer cells, a decrease in metastasis, or any combination of the above.

B. Detailed Description

The present invention involves the production of modified vectors, e.g., viruses, bacteria, yeast, protozoans, fungi or autonomously replicating genetic components such as plasmids, wherein the modification comprises the introduction and operable linkage of one or more stress gene regulatory elements to a gene that affects at least one of the replication, pathogenicity and/or immunogenicity of the vector. For example, a preferred embodiment of the invention will comprise the production of modified adenoviruses by the incorporation and operable linkage of at least one heat shock promoter sequence, e.g., HSP70 or HSP70B to a gene that affects replication, e.g., E1a or E1b.

By contrast, another preferred embodiment will comprise producing a vector having altered pathogenicity, e.g., Vibrio Cholera, by placing a cholera toxin gene under the regulatory control of a heat shock regulatory sequence, e.g., the HSP70 or HSP70B promoter. Thereby, a modified microorganism is produced that exhibits pathogenicity (expression of toxin) only under stress-induced conditions.

In still another preferred embodiment of the invention the immunogenicity of a vector, e.g., a protozoan, bacteria or virus will be modified by placing a gene encoding an immunodominant epitope, e.g., one that is essential for neutralization or protective immune response, under the regulatory control of a stress gene regulatory sequence.

Thereby, modified vectors are obtained wherein the replication, pathogenicity and/or immunogenicity is regulatable under conditions of cellular stress, such as infection, exposure to thermal or other radioactive, or other toxicity such as heavy metals.

The induction of stress inducible genes may occur in situ, e.g. at sites where stress conditions are endogenously present, e.g. tumor sites or sites of infection. Other instances where stress conditions may be endogenously present are patients on chemotherapy, e.g. cisplatin.

A significant novel and non-obvious application of the modified vectors of the invention is that they enable stress-induced expression of particular vector genes that modulate replication, pathogenicity and/or immunogenicity, thereby facilitating spatial, temporal and conditional regulation thereof. This is advantageous as the replication, pathogenicity and/or immunogenicity of the modified vector can e.g., be “turned on” at a desired target site, such as a tumor site or the site of an immunological disease or infection. For example, a modified adenovirus which has been modified by operable linkage of the E1a and/or E1b genes to heat shock regulatory sequences, such as a HSP70 or HSP70B promoter, can be administered to a tumor bearing subject and the replication capacity of the adenovirus selectively “turned on” at the tumor site by application of thermal stress.

Thermal stress can be effected by heating target tissues e.g., a tumor by application of ultrasound, laser, microwave, magnetic induction, or any other suitable form of heating or irradiation. Preferably, the application of thermal stress is applied very precisely at a certain site (spatial control) and at a certain time (temporal control) and under specific conditions, thereby inducing vector replication to occur selectively at the site and time when the thermal stress is being applied. An advantage of stress proteins such as heat shock proteins is that their expression is exquisitely sensitive to stress, e.g., temperature stress. By “exquisitely sensitive” is meant that the promoters are substantially silent unless stress inducible conditions are present. Consequently, the vector does not replicate unless exposed to conditions of stress.

As discussed previously, while in the preferred embodiment “stress conditions” will mean thermal stress, the invention also embraces the use of other types of stress that induce the expression of stress genes such as infection, and toxicity (e.g., chemical or heavy metal-associated toxicity). Those skilled in the art are well aware of conditions that result in the induction of stress genes. For example, alternative methods for induction of local or systemic stress induction include the addition of drugs, metal salts or other inducers of the stress response.

Preferred Vectors

While a variety of vectors may be used, it should be noted that viral vectors such as retroviral vectors are useful for modifying eukaryotic cells because of the high efficiency with which the retroviral vectors transfect target cells and integrate into the target cell genome. Additionally, the retroviruses harboring the retroviral vector are capable of infecting cells from a wide variety of tissues.

Retroviral vectors are produced by genetically manipulating retroviruses. Retroviruses are called RNA viruses because the viral genome is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site). See Mulligan, R. C. (1983) In:

Experimental Manipulation of Gene Expression, M. Inouye (ed), 155-173; Mann, R. et al. (1983) Cell, 33:153-159; Cone, R. D. and R. C. Mulligan (1984) Proceedings of the National Academy of Sciences, U.S.A., 81:6349-6353.

Retroviral vectors are particularly useful for modifying cells because of the high efficiency with which the retroviral vectors transduce target cells and integrate into the target cell genome. See Miller, A. D. (1992) supra. Retroviruses harboring the retroviral vector are capable of infecting dividing cells from a wide variety of tissues. These vectors have the ability to stably integrate the transferred gene sequences into the chromosomal DNA of target cells.

The design of retroviral vectors is well known to one of skill in the art. See Singer, M. and Berg, P. supra. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including European Patent Application EPA 0178220, U.S. Pat. No. 4,405,712, Gilboa (1986) Biotechniques 4:504-512; Mann et al. (1983) Cell 33:153-159; Cone and Mulligan (1984) Proc. Natl. Acad. Sci. USA 81:6349-6353; Eglitis, M. A. et al. (1988) Biotechniques 6:608-614; Miller, A. D. et al. (1989) Biotechniques 7:981-990; Miller, A. D. (1992) Nature, supra; Mulligan, R. C. (1993) supra; and Gould, B. et al. and International Patent Application NO. WO 92/07943 entitled “Retroviral Vectors Useful in Gene Therapy”. The teachings of these patents and publications are incorporated herein by reference.

In addition to the retroviral vectors mentioned above, cells may be transformed with adenoviruses or adeno-associated viral vectors. See, e.g., Methods in Enzymology, Vol. 185, Academic Press, Inc., San Diego, Calif. (D. V. Goeddel, ed.) (1990) or M. Krieger (1990), Gene Transfer and Expression—A Laboratory Manual, Stockton Press, New York, N.Y. and the references cited therein. Adenoviruses are double stranded, linear DNA viruses that cause the “common cold”, pneumonia, conjunctivitis and other illnesses. There are 42 serotypes of adenovirus known to infect humans.

Adenoviruses typically enter cells by receptor-mediated endocytosis. The specific receptor is unknown. Following internalization, the genome of the vector likely does not integrate into the host genome but instead functions episomally. This leads to only transient gene expression and also avoids random genome integration and its potential problems such as induced tumorigenicity.

Adeno-associated viruses (AAVs) require helper viruses such as adenovirus or herpes virus to achieve productive infection. In the absence of helper virus functions, AAV integrates (site-specifically) into a host cell's genome, but the integrated AAV genome has no pathogenic effect. The integration step allows the AAV genome to remain genetically intact until the host is exposed to the appropriate environmental conditions (e.g., a lytic helper virus), whereupon it re-enters the lytic life-cycle. Samulski (1993) Current Opinion in Genetic and Development, 3:74-80, and the references cited therein provides an overview of the AAV life cycle. See also West et al. (1987) Virology, 160:38-47; Carter et al. (1989), U.S. Pat. No. 4,797,368; Carter et al. (1993) WO 93/24641; Kotin (1994) Human Gene Therapy, 5:793-801; Muzyczka (1994) J. Clin. Invest. 94:1351, and Samulski, supra, for an overview of AAV vectors.

Recombinant AAV vectors (rAAV vectors) deliver foreign nucleic acids to a wide range of mammalian cells (Hermonat & Myzycka (1984) Proc. Natl. Acad. Sci. USA 81:6466-6470; Tratschin et al. (1985) Mol. Cell Biol. 5:3251-3260) integrate into the host chromosome (Mclaughlin et al. (1988) J. Virol 62:1963-1973) and show stable expression of the transgene in cell and animal models (Flotte et al. (1993) Proc. Natl. Acad. Sci USA 90:10613-10617). Moreover, unlike retroviral vectors, rAAV vectors are able to infect non-dividing cells (Podsakoffet al. (1994) J. Virol 68:565666; Flotte et al. (1994) Am. J Respir. Cell Mol. Biol. 11:517-521). Further advantages of rAAV vectors include the lack of an intrinsic strong promoter, thus avoiding possible activation of downstream cellular sequences, and their naked icosahedral capsid structure, which renders them stable and easy to concentrate by common laboratory techniques.

rAAV vectors have several properties which make them preferred gene delivery systems in clinical settings. They have no known mode of pathogenesis and 80% of people in the United States are currently seropositive for AAV (Blacklow et al. (1971) J. Natl. Cancer Inst. 40:319-327; Blacklow et al. (1971) Am. J. Epidemiol. 94:359-366). Because rAAV vectors have little or no endogenous promoter activity, specific promoters may be used depending on target cell type. rAAV vectors can be purified and concentrated so that muliplicities of infection exceeding 1.0 can be used in transduction experiments. This allows virtually 100% of the target cells in a culture to be transduced, eliminating the need for selection of transduced cells.

Plasmids designed for producing recombinant vaccinnia, such as pGS62 (Langford, C. L. et al. (1986) Mol. Cell. Biol. 6:3191-3199) may also be used. Finally, nonpathogenic vectors derived from HIV have been reported to transform non-dividing cells, and may also be used.

The modified vectors of the present invention, e.g. viral vectors, optionally also comprise selectable markers which result in nucleic acid amplification such as the sodium, potassium ATPase, thymidine kinase, aminoglycoside, phosphotransferase, hygromycin B phosphotransferase, xanthine-guanine phosphoribosyl transferase, CAD (carbaml phosphate synthetase, aspartate transcarbamylase, and dihydroorotase), adenosine deaminase, dihydrofolate reductase, and asparagine synthetasae and ouabain selection. Alternatively, high yield expression systems not involving nucleic acid amplification are also suitable, such as using a baculovirus vector in insect cells.

More Complete Listing of Viruses Applicable for Use in Producing Modified Vector

Suitable viruses for use in the invention include single stranded and double stranded RNA and DNA viruses, including human and animal pathogens such as those of the families Picornaviridae, Calciviridae, Togaviridae, Flaviviridae, Coronoviridae, Rhabdoviridae, Filoviridae, Paramyxoviridae, Bunyaviridae, Arenaviridae, Resviridae, Birnaviridae, Retroviridae, Hepadnaviridae, Parvoviridae, Papovaviridae, Adenoviridae, Herpesviridae, Poxviridae and kidoviridae.

Picornaviridae strains include those of genus enterovirus, cardiovirus, rhinovirus, apritovirus, such as poliovirus 1, 2, 3, coxsackieviruses A1-A22, A24, coxsackieviruses B1-B6, human echoviruses 1-9, 11-27, 29-37, human enteroviruses, human rhinoviruses 1-113, hepatitis A virus, swine vesicular disease virus, murine poliovirus, bovine enteroviruses 1-7, simian virus, bovine rhinoviruses 1-2, equine rhinoviruses 1-2, Aptasvirus and foot-and-mouth disease viruses.

Caliciviridae includes especially viral strains that cause gastroenteritis in humans, vesiculolar exonthema viruses 1-2, feline calciviruses and calciviruses of calves, swine, dogs, fowl and chimpanzees.

Togaviridae includes viruses of generic Alphavirus, Rubivirus, Pestivirus and Arterivirus. Human and animal strain pathogens include those that cause encephalitis.

Flaviviridae includes the genus Flavivirus. Human and animal Flaviviruses include strains that cause yellow fever, dengue viruses, West Nile viruses, St. Louis encephalitis virus, Japanese encephalitis virus, Murray Nally encephalitis virus, Roclo virus, tick-borne encephalitis virus, and others.

Coronoviridae includes those of genus Coronavirus, such as human pathogens that cause the common cold and upper respiratory tract infections, and animal pathogens that cause bronchitis and gastroenteritis.

Rhabdoviridae includes those of genera Vesiculovirus, Lyssavirus and others. Human and animal pathogens include vesicular stomatis, Chandipera virus, Piry virus, rabies virus, Mokola virus and Devonhage virus.

Filoviridae include those of genus Filovirus such as Marburg and Ebola viruses.

Paramyxoviridae include those of genera paramyxovirus, morbillivirus and pneumovirus, such as viruses that cause Parainfluenza 1, 2, 3, 4, mumps virus, Morbillivirus, measles virus, Pneumovirus, Newcastle disease virus, parainfluenza 1 virus, parainfluenza 3 virus, Respiratory Syncytial Virus and others.

Bunyaviridae includes those of the genera Bunyavirus, Phlebovirus, Nairovirus, Ukuvirus and Hantavirus, such as strains that cause sandfly fever and hemorrhagic fever.

Arenaviridae include human and animal pathogens such as LCM virus, Lassa virus, and Junin virus.

Reoviridae viruses include those of genera reovirus, orbivirus, rotavirus, phytoreovirus, fujivirus and cypovirus such as Reovirus 1, 2, 3, orbivirus, Epizootic Hemorrhagic viruses and rotaviruses.

Bimaviridae include bimavirus strains.

Retroviridae include those of genera Lentivirus, Spornavirus and others HTLV-I, HTLV-II, HIV-I, HIV-II, bovine leukosis virus, feline sarcoma and leukemia viruses, avian reticuloendotheliosis, mouse mammary tumor virus, visna viruses, equine infectious anemia virus, FIV, bovine lentivirus, SUV and others.

Hepadnaviridae include hepatitis B-like viruses, e.g. hepatitis B and other related viruses.

Parvoviridae includes those of genera parvovirus, dependovirus and densovirus, such as human and animal parvoviruses.

Adenoviridae include those of genera Mastadenovirus and Aviadenovirus, such human and animal mastadenovirus strains.

Herpesviridae include those of genera Simplexvirus, Varicellavirus, Betaherpesvirinae, Cytomegalovirus, lymphocryptovirus, Marek's disease-like viruses and Rhadinovirus. Examples are herpes simplex, varicella-zoster virus, human cytomegalovirus, Marek's disease virus, EB virus and others.

Poxviridae includes those of genera Orthopoxvirus, Chordopoxvirinae, Parapoxvirus, Avipoxvirus, Capripoxvirus, Leporipoxvirus, Suipoxvirus, and Entomopoxvirinae. Examples include human and animal vaccinia virus, variola (smallpox), monkeypox, cowpox, pseudocowpox, and others.

Iridoviridae include those of genera Iridovirus, Chloriridovirus, Ranavirus and Lymphocystivirus, such as African swine fever virus.

Bacterial Species Useful in Constructing Modified Vector According to the Invention

Suitable bacterial strains for use in the invention include by way of example, Escherichia coli, Bacillus subtilis, Streptococcus mutans, Pasteurella haemolytica, Neisseria gonorrhea, Mycobacterium tuberculosis, Haemophilus vaginalis, Group B Streptococcus ecoli, Microplasma hominis, Staphylococcus aureus, Staphylococcus epidermis, Streptococcus pyrogenes, Aerobacter aerogenes, Salmonella enteritidis, Pseudomonal aeroginosa, Klebsiella pneumoniae, Salmonella typhimura, Haemophilus influenzae, Streptococcus viridans, Proteus vulgaris, Shigella dysentariae, Streptococcus Group B, Diplococcus pneumoniae, Corynebacteria acnes type 1 and type 2, Haemophilus ducreyi, Granoloma inguinate, Lymphopatha venereum, Trepesoma pallidom, Brucella abortus, Brucella malitensis, Brucella sois, Brucella caris, Campylobacter fetus, Campylobacter fetus intestinalis, Leptospira pomona, Listeria monocytogenes, Brucella ovis, Chlamydia psittaci, Actinobacillus equuli, Salmonella abortus ovis, Salmonella abortus equis, Corynebacterium equi, Corynebacterium pyogenes, Actinobacillus seminis, Mycoplasma bovigenitalium, Clostridium tetani, Pseudomonas maltophiia, Streptococcus equisimili, Streptococcus uteris, Streptococcus bovis, Pasteurella multocida, Pasteurella haemolytica, Moratella bovis, Actinobacillus lignieresi, Clostridium difficile, Corynebacterium renate, Fusobacterium necrophoron, Bacillus cereus, Salmonella dublin, Salmonella heidelberg, Salmonella paratyphi and Yersinia enterocolitica. Bacterium strains can be isolated or obtained from commercially available sources, such as the American Type Culture Collection.

Listing of Suitable Yeast Vectors

Suitable yeast vectors for use in the invention include by way of example Saccharomyces, Pichia, Hanserula, Schizosaccharomyces, Candidas, Yarrowin, Klugueromyces, Cryptococcus, Rhodotorula. Preferred species include Saccharomyces cerevisiae, Saccharomyces carlbergensis, Candida albicans, Candida ketyr, Candida tropicalis, Cryptococcus laventii, Cryptococcus neoformons, Hansenula anomala, Hanserula polymorphna, Kluyveromyces fragilis, Kluyveromyces marxianus var. lactis, Pichia pastoris, Rhodotorula rubra, Schizosaccharomyces pombe, and Yarrowia lipolytica.

Production of Modified Vectors

In general, according to the invention, a desired vector is obtained that comprises at least one gene which affects vector replication, pathogenicity and/or immunogenicity, and the genome of such vector is modified so as to place the particular gene (or genes) under the regulatory control of at least one stress gene “regulatory sequence”.

Regulatory sequences according to the invention are intended to include any sequence that renders the gene stress-inducible, in it is substantially “off” under non-stress conditions and “turned-on” under stress conditions, e.g., elevated temperature. Such regulatory sequences include nucleic acid, peptide or other sequences that regulate transcription, translation and/or activity of stress proteins such as promoters, enhancer, suppression binding sites, binding domains, trafficking, and post-translational and signaling modification. Most typically, the regulatory sequence will comprise the promoter and/or enhancer of a stress gene, most preferably a heat shock promoter such as HSP70 or HSP70B.

Stress genes and heat shock regulatory sequences are well known in the art and are identified in references previously cited.

Additionally, genes that affect replication of various desired vectors, e.g., viruses, yeast, fungi, bacteria, protozoans or plasmids are known in the art or can be identified by gene knock-out or mutagenesis.

In the case of many viruses, genes which affect replication are well known. For example, the replication of adenoviruses is known to be controlled by a number of genes including the E1a and E1b early replication genes.

Similarly, genes and portions of the genome of retroviruses that affect replication of the pathogenicity of retroviruses such as lentivirus, HIV, F1V, HTLV-1, HTLV-II, and FeLV are known, i.e., such genes are comprised particularly in the LTR region, and include the U5 region, the U3 region, the R region, the PBS region, the AUG start codon region, the polyA region, RNA splice sites, the leader region, the TAT splice site, the ART splice site and the cap site. See U.S. Pat. No. 5,580,761 issued Dec. 3, 1996 to Greatbatch et al., incorporated by reference herein for a discussion of retroviral genes and sequences involved in replication.

Portions of the hepatitis viral genome that affect replication and/or pathogenicity are also known and include HBV polymerase, and immediate early genes (e.g., IE 4 and 5 genes) (See respectively e.g., U.S. Pat. No. 5,610,050 issued Mar. 11, 1997 to Blum et al., and Smith et al., Proc. Natl. Acad. Sci., USA 83:2787-2791 (1986)).

Also, genes that affect the replication of adeno-associated virus are known and include the AAV rep genes (See U.S. Pat. No. 5,837,484 incorporated by reference herein). Examples thereof include Rep 78, Rep 40, Rep 52 and Rep 68.

Further, genes that affect the replication of various poxviruses, are also known. Poxviruses include e.g., vaccinia virus (which is well used as a vaccine vector), pseudorabies, swine poxvirus, as are genes that affect the replication of many other DNA viruses such as herpes, papova, papilloma and parvoviruses. Methods for identifying viral genes that are essential and affect viral replication are well known in the art. (See e.g., U.S. Pat. No. 5,665,362 issued Sep. 9, 1997 which discloses the production of attenuated viruses and the identification of essential genes by mutagenesis of different parts of the viral genome).

Additionally, it is well known that certain vector genes, e.g., viral or bacterial genes encode “immunodominant” epitopes that are essential in elicitation of a protective or neutralizing immune response. Typically, such genes correspond to proteins that are on the surface of the vector, e.g. a bacterium or virus, e.g., viral capsid proteins. Examples thereof include the “VP” genes of many viruses, e.g., the VP1, VP2, VP3 genes of chicken anemia virus, and the L1 and L2 genes of papillomavirus, et al.

Similarly, genes or sequences that affect the replication of other vectors according to the invention are known or can be identified by an ordinary artisan. For example, in the case of yeast, there are various genes known to affect cell cycle and proliferation such as the CDC genes, RAD genes (e.g., RAD9, RAD21, RAD17) and MEC-1, MEC-2 and MEC-3 genes.

Also, genes that affect the proliferation of bacterial cells are known as DNAs that affect the replication of extrachromosomal elements (plasmids) such as autonomous replication sequences (ARS's).

Therefore, once an appropriate vector is selected, e.g. a virus having known application for vaccine or gene therapy, the genome of such vector will be modified such that a gene that regulates replication, immunogenicity or pathogenicity is modified such that the endogenous promoter is replaced by a stress protein regulatory sequence, e.g., a heat shock promoter. This can be effected by well known methods for inserting a desired sequence at a target genomic site, e.g. via homologous recombination.

For example, this may be accomplished by operably linking the gene of a vector, e.g. a bacteriol gene that encodes a protein or indirectly results in expression of a protein involved in pathogenic phenotype (e.g., cholera toxin, or metabolism of a pre-molecule to a toxic compound) to a desired heat shock promoter such as HSP70B.

In another embodiment, this may be effected by operably linking a gene contained in a vector, e.g. adenovirus gene that is involved in replication, e.g., E1a or E1b to a heat shock promoter, e.g. HSP70. This is accomplished by integrating the HSP70 promoter such that it replaces the endogenous promoter of the E1a or E1b gene, thereby resulting in a vector (recombinant virus) having its replication capacity temperature inducible.

In yet another embodiment of the invention, this can be accomplished by obtaining a vector, e.g. a virus, having a gene that affects immunogenicity, e.g. a gene encoding an immunodominant epitope such as the L1 gene of papillomavirus and replacing the endogenous promoter of said gene with a stress protein regulatory sequence.

Suitable protozoan vectors for use in the invention include those of the genera Plasmodia, Trypanosome and others, especially those that infect humans and agricultural animals. Preferred protozoans include those that cause malaria (e.g., Plasmodium falciparum, Plasmodium vivax).

As noted above, the invention further embraces the incorporation of additional genes, especially those encoding an active agent into the modified vector, e.g. a therapeutic enzyme, cell-affecting agent, drug, diagnostic agent or cytotoxic agent, or the use of the subject vectors in combination with other therapies, e.g. neoplastic agents, cytotoxins, drugs, small molecules, cytokines, therapeutic enzymes, hormones, antibodies, lymphokines, antisense oligonucleotides, ribozymes, radioimmunotherapy, external beam radiation, etc.

The nature of the specific alternative therapy will depend upon the particular disease being treated, the prognosis of the patient, and any other treatments.

In particular, the invention embraces the modification of the particular vector to include or modulate cell-affecting genes such as genes that encode apoptopic inducers, genes that affect cell death, aging, division and DNA synthesis, mitochrondial genes, peroxisomal genes, immunosuppressant genes, ATP-binding proteins, cytoskeletal genes, all rescue genes, genes involved in cell damage and repair. A listing of potential yeast and mammalian genes which may be included in the vector is provided below.

Yeast Genes

CELL RESCUE, DEFENSE, CELL DEATH AND AGING PRE3, PRE1, PUP2, RPN12, RPT1, MAG1, OGG1, SED1, ATH1, SPE2, GRE3, TPS2, TPS1, ATR1, ATX1, SK13, SK12, SK18, APN1, HPR5, ERG5, CCZ1, SRA1, SNF1, YCK1, YCK2, HRR25, CTA1, CTT1, WSC4, PAM1, TIR2, TIR1, HDF2, TFB4, RAD1, HAM1, LYS7, SOD1, KIN28, DIT2, ERG11, CYC7, CCP1, PHR1, DAK2, DAK1, ALR1, ALR2, HOR2, RAD17, DDC1, DDR2, ALK1, HEL1, SSL2, RAD5, SGS1, PIF1, RAD3, CDC9, REV7, NTG1, RAD18, RAD57, RAD55, XRS2, RAD30, MMS21, RAD51, RAD10, PS02, REV1, DIN7, RAD54, CDC2, PES4, POL2, REV3, RPB7, RPB4, SGE1, UBA1, UBC4, UBC5, RAD6, QR18, RNC1, NTG2, ERC1, RAD4, ETH1, FKB2, YHB1, FLR1, MEC3, ZWF1, GSH1, GRX1, TTR1, HYR1, GLR1, YCF1, FPS1, GPD1, RAS2, RAS1, CUPS, HSP26, HSP30, HSP12, HSP104, DDR48, HSC82, HSP82, MDJ1, MDJ2, HSP60, HSP78, ECM10, SSE1, SSA1, SSA3, SSA4, SSA2, SSE2, HSF1, HIG1, HDF1, HMS2, GRE1, DD11, RTA1, SIMI, LAG2, ZDS1, MET18, SNG1, NCA3, KTI12, UTH1, SUN4, SSU81, SSD1, TH14, KAR3, LIF1, SFA1, LAG1, LTV1, MDR1, SSK22, SSK2, HOL1, CIS3, HSP150, PIR3, MAC1, CUP1 A, CUP1 B, YDJ1, SSQ1, SSC1, IMP2, MPT5, ATX2, SN02, MLP1, NHX1, NCP1, NSR1, SNF4, RAD16, RAD7, RAD14, RAD23, ROD1, MGT1, OSM1, SIP18, SAT2, MNR2, MMS2, PNT1, CYP2, PAD1, PDR5, PDR3, PDR6, RTS1, PA13, HOR7, DUN1, IRE1, MKK2, MET22, PPZ2, PTC1, PTP2, MMS4, RAD52, PDR13 SLG1, GRR1 HIT1, RDH54 BR01, PIR1 MSRA, RNR4 RNR3, HAL1, YGP1, CDC55, PPZ1, PKC1, HAL5, MKK1, HOG1, SLT2, BCKi, RAD53, SIR4, SIR3, SIR2, MGA1, FUN30, YR02, DNL4, RRD1, SAT4, RAD27, MSN2, ST11, PAU3, PAU2, PAU5, PAU1, PAU4, PAU6, (MLP1), RAD2, FZ_F1, SSU1, SOD2, CRS5, BCK2, ASM4, TIP1, TFB1, CCL1, SSL1, TFB3, TFB2, TSAI, TRX1, TRX2, ROX3, PDR1, GTS1, MCM1, SKN7, CAD 1, MSN4, YAP1, SLN1, SSK1, PBS2, UB14, RSP5, SVS1, ZRC1

CELL GROWTH, CELL DIVISION AND DNA SYNTHESIS GSC2, PLC1, PRE3, PRE2, PRE1, PUP2, RPN12, RPT6, RPT1, DIS3, RP SOA, AGA1, AGA2, ASG7, ACH1, ACT1, SAC6, ARP100, ABP1, PAN1, ARP 2, AREIARE2, SPE2, CYR1, SRV2, ADK2, GCS1, SOH1, TUB1, TUB3 SAG1, AKR1, YAR1, SK18,

ARG82, ABF1, STE6, BAR1, 8011, TUB2, RBI-2, BIG1, BI M1, BAT1, BEM1, BEM4, SBE2, BN14, BUD6, 8012, BUD9, BUD4, BUD8, RC K2, CMK1, CNA1, CMP2, CNB1, CCH1, CMD1, SRA1, YCK1, YCK2, HRR2 5, CKA2, CKA1, YCK3, EST2, TFS1, SCM4, GIC2, GIC1, CAK1, BUB2, B UB3, ESR1, RAD24, DBF20, PDS1, HPC2, NUD1, CDC47, CDC10, CDC13, C DC37, CDC1, CDC40, CDC4, CDC20, CDC6, CDC46, CDC3, KAR1, BB P1, CDC50, FUS1, KRE9, EGT2, ARP1, CHS1, CHS2, CHS3, CHS5, MS11, CAC2, R LF2, CHL4, SMC1, SMC2, CIN1, SNF7, CLC1, COF1, PAM1, LAS17, HDF2, SEC3, SNF2, SWI1, SNF5, SNF1 1, DOC1, APC2, APC5, TAP42, CDC53, KAR 9, CCE1, CLB6, CLB5, CLN3, PCL2, CLN1, PCL1, CLN2, CI-133, CI-131, CLB 4, CI-132, FAR1, CKS1, CDC28, PH085, KIN28, SSN3, CLG1, DIT2, SLA1, SLA2, SP020, DPP1, RAD17, DDC1, HEL1, DNA2, RAD5, SGS1, HCS 1, PIF1, CDC9, MSH3, MSH6, MLH1, PMS1, MSH2, MSH1, POL4, REV 7, MRE11, RAD26, RAD9, RAD18, RAD57, RAD55, XRS2, MMS21, RAD51, RADIO, RAD 50, RFA3, RFA2, RFA1, RFC4, RFC5, RFC3, RFC2, RFC1, FOB1, TOP1, TO P2, TOP3, RAP1, RAD54, PR12, PR11, POL1, POI-12, CTF4, HUS2, CDC2, P ES4, POL2, DPB2, DPB3, MIP1, REV3, SSN8, GAL11, RGR1, SRB6, RP041, SEC59, DIP2, CDC14, MSG5, DYN1, UBC4, UBC9, CDC34, UBC5, UBC 1, UBC6, RAD6, QR18, ELC1, RNC1, CTS1, KEX2, APG1, SSP1, SUP35, EXM2, S PR1, EXG1, EXG2, DHS1, CAP1, CAP2, BRN1, GPR1, GIF1, MEC3, TU B4, CIS2, LTE1, SDC25, SRM1, CDC25, ROM2, BUDS, ROM1, SPT16, CDC43, G IP1, SIN4, SNF6, KRE6, GFA1, NGR1, WH12, RSR1, CIN4, RAS2, RAS1, GP A1, STE4, STE18, CDC42, MDG1, SEC4, TEM1, RH03, RH04, RI-102, RHO 1, CDC24, BEM2, BUD2, BEM3, LRG1, GPA2, SIS1, HSP82, HSF1, ABF2, HDF1, HDR1, RPD3, HSL7, HO, SBA1, HPR1, IDS2, NFI1, CSE2, MDM 1, MI-1 131, MIDI, SIMI, HIR3, SIS2, MAKI 1, LAS1, SPA2, WH14, ECM33, SET1, CTF19, CIN2, MCM16, SLK19, CYK2, CNM67, SST2, DPB11, DOS2, D FG16, AFR1, ZDS1, SR07, PEA2, FAR3, SMP2, WH13, CDC5, MET30, SAS2, SCC2, CIS 1, STN1, UTH1, PAC2, SSD1, SRP1, KRE5, KIP1, CIN8, SMY1, KIP2, KAR3, KIP3, CBF1, CBF2, SKP1, CEP3, CTF13, DBR1, LAG1, MIH1, BFR1, DIG2, DIG1, MFA1, MFA2, MFAlpha1, MFAIpha2, MID2, SSF1, MATALPHA2, MATA LPHA1, ALPHAI, ALPHA2, A2, A1, SAN1, PGD1, SPO11, MSH5, DMC 1, ISC10, MSH4, SP013, NDT80, REC104, HOPI, RED1, SP07, MUM2,ME15, S AE2, NAM8, REC107, REC102, REC114, MER1, RIM01, NDJ1, CDC54, CP R7, SYG1,MCM2, CIS3, HSP150, ACE2, CDC48, ASE1, YTM1, HSM3, YD J1, ERV1, FUS3, JNM1, MCD1, MMC1, MSB1, MSB2, MPT5, ZDS2, MSN5, KEM1, MLC1, MY02, MY04, MY05, MY03, MY01, DEC1, PMD1, M DS3, ASH1, UME1, UME6, NHP6A, RFT1, TRF5, NNF1, NDC1, BIK1, KAR2, KAR5, NUM1, CDC39, MAK16, NAP1, RAD16, RAD23, NBP35, ORC1, ORC6, ORC5, ORC4, ORC3, RRR1, SIC 1, BUD3, PWP2, STE3, STE2, OPY2, STE50, STE5, PEL1, TOR1, TOR2, PIK1, STT4, MSS4, SP014, POL32, IME4, SHP1, PDS5, FEN1, CSE1, FL08, PFY1, PHB2, PHB1, POI-30, AXL1, STE23, RAD28, CDC7, SMP3, MKK2, CDC15, ARD1, CHU, PPH3, PPH21, PPH22, PTC1, SE C9, PPS1, PTP3, YVH1, PTP2, PUS4, PCH2, PCH1, CBF5, SEF1, MMS4, SHR5, RAD59, RAD52, RHC18, RGP1, RVS167, RIM9, BNR11, BN11, SPT3, SOK2, KAR 4, DBF4, SDS22, MCM3, CTF18, SR04, SPH1, FUS2, MOB1, FL08, FIG1, FIG2, END3, DFG5, CTR9, TOM1, POP2, GRR1, SCP160, SUR1, MUM3, ZIP2 CDC45, RDH54, SHE3, SHE2, SHE4, GPI1, MIF2, ESP1, HOP2, DNA43, SMC3, PAC11, PAC10, RD11, RGA1, RNR1, RNR2, RNR4, RNR3, PRPS 1, RPL10, RPS1A, MTF1, SN12, CDC12, CDC11, SPR28, CDC55, GLC7, PKC1, GI N4, SPS1, RCK1, BUB1, IME2, YAK1, YPK2, RIM11, CLA4, MKK1, MEK1, I PL1, SGV1, SLT2, KSS1, BCK1, STE11, STE20, DBF2, HSL1, NRK1, SIT4, T PD3, ELM1, MCK1, RAD53, STE7, SWE1, MPS1, SAS3, HST1, SIR4, SIR3, SIR1, SIR2, CTH1, DOM34, HST4, RVS161, DNL4, IQG1, FUN16, HYM1, RT S2, MNN10, PRK1, MCM6, SAP155, SAP4, SAP190, SAP185, MUD13, M AD1, CIK1, NUF1, SPC97, SPC42, SPC98, CDC31, NUF2, MAD3, MAD2, DI T1, YSW1, SP012, SP016, MCD4, BDF1, SGA1, GSG1, SHC1, CDA1, CD A2, SMK1, SPS2, SPR6, SLZ1, SPS4, SPR3, SPS100, SPS18, RAD27, SNZ 1, SUR4, ST11, SBE22, CSE4, BMH 1, SVL3, SCH9, (MLP1), SSF2, RAD2, CDH1, CDC27, CDC26, CDC23, CDC16, APC1, APC 11, APC4, APC9, SAP30, RSC6, RSC8, STH1, SFH1, SAS5, JSN1, BMH2, SMT4, BCK2, HOC1, ZIP1, UFE1, EST1, TEL1, ANC1, CCL1, DST1, TRX1, TRX2, TRF4, PAT1, SPT4, SP T6, CDC36, SWI5, SWI4, PHD1, SWI6, GTS1, MCM1, IME1, SKN7, MBP1, SW13, SIN3, STE12, CIN5, SDS3, SP01, MOT2, RPG1, PRT1, CDC33, TPM1, TPM2, TWF1, TEC1, TTP1, STE13, PRP8, UB14, DSK2, RSP5, D OA4, UNG1, VPS45, VAN1, VRP1, DFG10, YHM2, GL′Q3, SFP1, STE24, RME1, SAE3, ME14, NHP6B, MOB2, EST3, RIM1

HEAT SHOCK PROTEINS CAT5, CPH1, CTT1, CYP2, DDR2, FPR2, HSC82, HSP104, HSP12, HSP150, HSP26, HSP30, HSP42, HSP60, HSP78, HSP82, KAR2, MDJ1, SIS1, S OD2, SSA1, SSA2, SSA3, SSA4, SSB1, SSB2, SSC1, SSE1, SSE2, ST11, TIP 1, TPS2, UB14, YDJ1

MITOCHONDRIAL AAC1, AAC3, AAT1, ABC1, ABF2, AC01, ACR1, ADH3, ADK2, AEP2, AFG3, ALD1, ALD2, ARG11, ARG2, ARG5,6, ARG7, ARG8, ARH 1, AT M 1, ATP1, ATP10, ATP11, ATP12, ATP14, ATP15, ATP16, ATP2, ATP3, ATP4, A TP5, ATP6, ATP7, ATP8, ATP9, BAT1, BCS1, CBP1, CBP2, CBP3, CBP4, CB P6, CBR1, CBS1, CBS2, CCA1, CCE1, CCP1, CEM1, CIT1, CIT3, COB, CO Q1, COQ2, COQ3, COQ6, COR1, COT1, COX1, COX10, COX11, COX12, C 0X3, COX14, COX15, COX17, COX2, COX3, COX4, COX5A, COX5B, COX6, COX7, COX8, COX9, CPR3, CTP1, CYB2, CYC1, CYC2, CYC3, CYC7, CYT1, CYT2, DB156, DLD1, DTP, ENS2, ERV1, FLX1, FUM1, GCV1, GCV3, GI-04, GPD2, GSD2, GUT2, HEM1, HEM15, HSP10, HSP60, HSP78, HTS1, IDH1, ID H2, IDP1, IFM1, ILV1, ILV2, ILV3, ILV5, ILV6, IMP1, IMP2, INH1, ISM1, KG D1, KGD2, LAT1, LEU4, LIP5, LPD1, LYS12, LYS4, MAE1, MAM33, MAS1, MAS2, MBA1, MCR1, MDH1, MDJ1, MDJ2, MDM10, MDM12, MEF1, MEF2, MET13, MGE1, MGM101, MIP1, MIR1, MIS1, MMM1, MMTi, M MT2, MOD5, MOL1, MRF1, MRP1, MRP13, MRP17, MRP2, MRP20, MRP21, MRP4, MRP49, MRP51, MRP8, MRPL10, MRPL 11, MRPL13, MRPL15, MRPL16, MRPL 17, MRPL 19, MRPL2, MRPL20, MRPL23, MRPL24, MRPL25, MRPL27, MRPL28, MRP L3, MRPL31, MRPL32, MRPL33, MRPL35, MRPL36, MRPL37, MRPL38, MR PI-39, MRPL4, MRPL40, MRPL44, MRPL49, MRPL6, MRPL7, MRPL8, M RPL9, MRPS28, MRPS5, MRPS9, MRS1, MRS11, MRS2, MRS3, MRS4, MRS5, MSD1, MSE1, MSF1, MSH1, MSK1, MSM1, MSP1, MSR1, MS S1, MSS116, MSS18, MSS51, MST1, MSU1, MSW1, MSY1, MTF1, M T01, NAM1, NAM2, NAM9, ND11, NHX1, NUC1, OM45, ORFA04514, OSM1, OXA1, PDA1, PDB1, PDX1, PEL1, PET111, PET112, PET117, PET122, PET123, PET127, PET130, PET191, PET309, PET494, PET54, PET56, PET9, PETCR46, PI-1131, PHB2, PIF1, PIM1, POR1, POR2, PPA2, PSD1, PUT1, PUT2, QCR10, QCR2, QCR6, QCR7, QCR8, QCR9, RCAi, RF2, RIM 1, RIM2, RIP1, RML2, RNA12, RPM2, RP 041, SC01, SCO2, SDI-11, SDH2, SDI-13, SDI-14, SECY, SHM1, SHY1, SLS1, SMF2, SOD2, SOM1, SSC1, SS©1, STF1, STF2, SUN4, SUV3, TIM17, TIM22, TIM23 TIM44, TIM54, TOM20, TOM22, TOM37, TOM40, TOM6, TOM7, TOM 70, TOM72, TRM1, TUF1, UNG1, VAR1, YAH1, YAL011W, YAT1, YBL013 W, YCR024C, YDR041W, YDR115W, YDR116C, YER073W, YFH1, YGLO68W, YGR257C, YHM1, YHR075C, YHR148W, YJL200C, YJR113C, YKLO55C, YKL120W, YKL134C, YKL192C, YLR168C, YMC1, YMC2, YML025C, YMR188C, YMR31, YNL081 C, YNL306W, YNR036C, YNR037C, YOR221 C, YPL013C, ETF-BETA

PEROXISOMAL CAT2, CIT2, CTA1, DAL7, EHD1, EHD2, FAA2, FAT2, FOX2, ICU, IDP 3, MDH3, MLS1, PEX11, PEX12, PEX13, PEX14, PEX17, PEX2, PEX3, PE X4, PEX6, PEX7, PEXB, POT1, POX1, PXA1, PXA2, SPS19, YBR204C, YDR 449C, YHR180W DNA-ASSOCIATED A1, A2, ABF1, ABF2, ADA2, ADE12, ADR1, ALPHA1, ALPHA2, ANC1, APN1, ARGR1, ARGR2, ARGR3, ARR1, ASH1, AZF1, BAS 1, BDF1, BR F1, BUR6, CAC2, CAD1, CAF17, CATB, CBF1, CBF2, CCE1, CCR4, CDC13, CDC36, CDC39, CDC46, CDC47, CDC54, CDC6, CDC7, CDC73, CDC9, CEF1, CEP3, CHA4, CHD1, CHU, CHL4, CRZ1, CSE1, CSE2, CSE4, CTF13, CUP2, CUP9, DAL80, DAL81, DAL82, DAT1, DBF4, DMC1, DNA2, DNA43, DNL4, D OS2, DOT6, DP131 1, DPB2, DPB3, DST1, ECM22, ENS2, EST1, EZL1, FCP1, FHL1, FKH1, FKH2, FL08, FZF1, GAL11, GAL4, GAT1, GBP2, GCN4, GCNS, GCR1, GCR2, GLN3, GL03, GTS1, GZF3, HAC1, HAP1, HAP2, HAP3, HAP4, HCM1, HDA1, HDF1, HFM1, HHF1, HHF2, HH01, HHT1, HHT2, HM01, HMS1, HMS2, H0, HOP1, HPR1, HPRS, HSF1, HTA1, HTA2, HTA3, HTB1, HT 62, IFH1, IME1, IME4, IN02, IN04, IXR1, KAR4, LEU3, LYS14, LYS20, LYS21, M AC 1, MAGI, MAL13, MAL23, MAL33, MATALPHA1, MATALPHA2, MBP1, MCD1, MCM1, MCM2, MCM3, MCM6, MED6, MER2, MET18, MET28, MET30, MET31, MET32, MET4, MGA2, MGT1, MIF2, MIG1, MIG2, MIP1, MLH 1, MOL1, MOT1, MPT4, MRE11, MSH1, MSH2, MSH3, MSH4, MSHS, MS11, MSN1, MSN2, MSN4, MTF1, NBN1, NC132, NDJ1, NGG1, NHP2, NHP6 A, NHP6B, NOT3, NUC2, OAF1, OP11, ORC1, ORC2, ORC3, ORC4, ORCS, ORC6, PAF1, PCH1, PCH2, PDR1, PDR3, PGD1, PHD1, PH02, PH04, PHR1, PIF1, PIP2, PMS1, POB1, POL1, POL12, POL2, POL3, POL30, PO L4, POP2, PPR1, PRI1, PR12, PS02, PUT3, RAD1, RAD10, RAD14, RAD 16, RAD18, RAD2, RAD23, RAD26, RAD27, RAD3, RAD4, RADS, RAD50, RAD51, RAD52, RAD54 RAD55, RAD57, RAD6, RAD7, RAP1, RAT-1, RCS1, REB1, REC102, RE C104, REC114, RED1, REG1, RET1, REV3, RFA1, RFA2, RFA3, RFC1, RFC2, RFC3, RFC4, RFCS, RGM1, RGT1, RIF1, RIF2, RIM1, RIM101, RLF2, RLM1, R ME1, RMS1, ROX1, ROX3, RPA12, RPA135, RPA14, RPA190, RPA34, RPA43, RPA49, RP131 0, RPB11, RPB2, RP133, RP134, RPBS, RPB6, RPB7, RPBB, RPB9, RPC10, RPC19, RPC25, RPC31, RPC34, RPC40, RPC53, RPC82, RPD3, RP021, RP031, RP041, RRN10, RRN11, RRN3, RRNS, RRN6, RRN7, RRN9, RSC4, RSC 6, RSC8, RTG1, RTG3, SASS, SEF1, SET1, SFH1, SFL1, SGS1, SIG1, SIN 3, SIN4, SIP2, SIP4, SIR1, SIR2, SIR3, SIR4, SKN7, SK01, SMC1, SMC 2, SMP1, SNF2, SNFS, SNF6, SOK2, SPKi, SPOL, SPS18, SPT10, SPT 15, SPT16, SPT2, SPT21, SPT23, SPT3, SPT4, SPT5, SPT6, SPTB, SRI 32, SRB4, SRBS, S RB6, SRB7, SR138, SR139, SSL2, SSN3, SSN6, SSNB, SSU72, STB4, STBS, S TE12, STH1, SUA7, SWI1, SW13, SW14, SW16, SWP73, TAF19, TAF25, TBF1, TEA1, TEC1, TFAI, TFA2, TF131, TF132, TF133, TFB4, TFC1, TFC2, TF C3, TFC4, TFCS, TFG1, TFG2, TH12, TOA1, TOA2, TOP1, TOP2, TOP3, TRF4, TS P1, TUP1, TYE7, UGA3, UME6, UNGI, USV1, XRS2, YAL019W, YAP1, YA P3, YAPS, YBL054W, YBR026C, YBR150C, YBR239C, YCR106W, YDR026 C, YDR060W, YDR213W, YER045C, YER184C, YFL052W, YIL036W, YIL 130W, YJL103C, YJL206C, YKL005C, YKL222C, YKR064W, YLL054C, YLRO 87C, YLR266C, YNL206C, YOL089C, YOR172W, YOR380W, YOX1, YPL133C, YPR008W, YPR196W, YRR1, ZAP1, ZIP1, ZU01

IMMUNOSUPPRESSANT FEN1, SSH4, SHR3 CYCLINS CCU, CLB1, CLB2, CLB3, CL134, CLBS, CLB6, CLG1, CLN1, CLN2, CLN 3, CTK2, PCL1, PCL10, PCL2, PCLS, PCL6, PCL7, PCLB, PCL9, PH080, S SNB, YBR095C

ATP-BINDING CASSETTE PROTEINS ADP1, ATM 1, CAF16, GCN20, MDL1, MDL2, PDR10, PDR11, PDR12, PDR15, PDRS, PXA1, PXA2, SNQ2, STE6, YBT1, YCF1, YDL223C, YD R091C, YEF3B, YER036C, YHL035C, YKR103W, YKR104W, YLL015W, YNR070W, YOR011W, YOR1, YPL226W

CYTOSKELETAL ABP1, ACF2, ACT1, AFR1, AIP1, AIP2, ARP3, AUT2, AUT7, BEM1, BI M1, BN11, BN14, BUD3, BUD6, CAP1, CAP2, CDC10, CDC11, CDC12, CDC 3, CIN1, CIN2, CIN4, CMD1, COF1, CRN1, END3, GIC1, GIC2, GIN4, J NM1, KAR9, KIP2, KIP3, LAS 17, MDM1, MHP1, MY01, MY02, MY03, MY04, MY05, PFY1, RVS161, RVS167, SAC6, SAC7, SEC 1, SHE3, SHM2, SLA1, SL A2, SMY1, SMY2, SPA2, SPH1, SPR28, SPR3, SRV2, TCP1, TPM1, TPM2, TUB1, TUB2, TUB3, VPS16, VRP1 APOPTOSIS ATP1, ATP14, ATP15, ATP16, ATP2, ATP3, ATP4, ATPS, ATP6, ATP7, ATP8, ATP9, CYC1, SH01, SSK2, SSK22, SW13, SXM1

CELL RESCUE ACC1, ALD6, BCK1, BEM 1, BEM2, BIM1, BMH1, BMH2, CAN1, CBF1, CDC1, CDC14, CDC15, CDC20, CDC25, CDC28, CDC33, CDC37, CDC 42, CDC43, CDC53, CDC6, CHC1, CIN8, CKA1, CKA2, CLA4, CLB1, CLB2, CLB3, CLB4, CLB5, CLN1, CLN2, CLN3, CMP2, CNA1, COF1, CTT1, DBF2, DBF20, DPM1, ERG25, GIC1, GIC2, GPA1, GRR1, HCA4, HIS4, HOC1, HSF1, KAR1, KES1, KRE6, KSS1, MBP1, NMT1, ORC2, ORC5, PDE2, PEP12, PEP7, PKC1, P LC1, PMR1, POL30, PRP18, RAM1, RAS1, RAS2, RBL2, RED1, RFC1, RH01, RH03, RH04, SAC1, SEC13, SEC14, SEC22, SEC4, SET1, SIS2, SKP1, SPC98, SRA1, SR04, SRP1, SSA1, SSA2, SSA4, SSN8, STE20, STN 1, STT4, SUJ 3, SWE1, SW14, SW16, TEL1, TOR1, TUB1, TUB4, VMA1, YCK1, YCK2, YPT1

CELL DAMAGE APN1, BUB1, CDC28, CDC45, CDC46, CDC47, CDC54, CDC7, CLB1, CLB2, CLB3, CLB5, DDC1, DDR2, DDR48, DIN7, DUN1, ECM32, HSM3, IMP2, MEC1, MEC3, MGT1, MOL1, MRE11, MUS81, NTG1, PDS1, PGD1, P HR1, POL2, POL3, POL30, POL4, PR11, PS02, RAD14, RAD16, RAD17, RAD18, RA D24, RAD30, RAD51, RAD52, RAD54, RAD55, RAD57, RAD7, RAD9, RDH54, REV3, RFA1, RFC5, RNR1, RNR2, RNR3, RNR4, RPH1, SIC1, SML1, SP K1, STN1, STS1, TEL1, TFA1, TFA2, TUP1, UBC7, UB14, XBP1, YBR098W, YFH1

OTHER RELEVANT MUTANTS AND GENES Y-1,9520b, C658-K7, JPD 4, JPM 9, Cy32, E354, JC488, PSY 142, 01-2, Y217, JC787-9A, ML1-21, Y500,86-9C, GL1, GT5-1A, HD565A, PZ1, 127-4D, Y229, JC302-26B, JC482, LB2211-2B, MH41-7B/P21, erg 81, SEY6211, GL4, K335, MK20, MK34, DE4-3A, DE4-3B, DE4-3C, MMY011, UH 1-GRGZ, 2150-2-3a, Y211, DP1/517,943,1117, C658, 1252, H79.20.3, LB1-3B, C658-K42, R29B, LB54-3A, XW520-9A, ade7, D225-5A, 309, SDH1, SDH2, SDH3, SDH4, TCM62, PDE1, PDE2

Mammalian Genes

11-beta hydroxysteroid dehydrogenase type II, 12-lipoxygenase, 17-beta hydroxysteroid dehydrogenase, 60S ribosomal protein L6,6-Omethylguanine -DNA methyltransferase, Activating transcription factor 2, Activating transcription factor 3, Activating transcription factor 4, Activin beta E, Activin receptor type 11, Acyl-CoA dehydrogenase, Acyl CoA Carrier Protein, Adenine nucleotide translocator 1, Alanine aminotransferase, Alcohol dehydrogenase 1, Alcohol dehydrogenase 2, Alcohol dehydrogenase 3, Alcohol dehydrogenase 4, Alcohol dehydrogenase 5, Aldehyde dehydrogenase 1, Aldehyde dehydrogenase 2, Aldehyde dehydrogenase 3, Alpha 1-antitrypsin, Alpha-1 acid glycoprotein, Alpha-1 antichymotrypsin, Alpha-catenin, Alphatubulin, Apolipoprotein A1, Apolipoprotein A11, Apolipoprotein Clil, Apolipoprotein E, Aryl hydrocarbon receptor, Aspartate aminotransferase, mitochondrial, Ataxia telangeictasia, ATP-dependent helicase 11 (70 kDa), ATP-dependent helicase 11 (Ku80), BAG-1, BAK, Bax (alpha), Bcl-2, Bcl-xL, Beta-actin, Bilirubin UDP-glucuronosyl-transferase isozyme 1, Bilirubin UDP-glucuronosyl-transferase isozyme 2, Biliverdin reductase, Branched chain acylCoA oxidase, BRCA1, BR-cadherin, C4bbinding protein, c-abl, Calcineurin B, Calnexin, Calprotectin, Calreticulin, canalicular multispecific organic anion transporter, Carbonic Anhydrase 111, Carnitine palmitoyl-CoA transferase, Caspase 1, Caspase 2 (Nedd2), Caspase 3 (CPP32beta), Caspase 5 (ICE relIII), Caspase 6 (Mch2-alpha), Caspase 7 (Mch3alpha), Caspase 8 (FLICE), Catalase, CatecholOmethyltransferase, CCAAT/enhancer-binding protein alpha, CCAAT/enhancer-binding protein epsilon, Cell division cycle protein 2, Cell division cycle protein 20, Cell division cycle protein 25, Cellular retinoic acid binding protein 1, Cellular retinoic acid binding protein 2, cerb; c-fos, Checkpoint kinase-1, Cholesterol esterase, c-H-ras, cjun, Clusterin, c-myc, Complement component C3, Connexin 30, Connexin32, Connexin-40, Corticosteroid binding globulin, Corticotropin releasing factor, C-reactive protein, Creatine kinase b, Cyclin D1, Cyclin dependent kinase 1, Cyclin dependent kinase 4, Cyclin dependent kinase inhibitor 1A, Cyclin E, Cyclin G, Cyclin-dependent kinase 4 inhibitor (P116), Cyclindependent kinase 4 inhibitor B (P16), Cyclin-dependent kinase inhibitor P27Kip1, Cyclooxygenase 2, Cystic fibrosis transmembrane conductance regulator, Cytochrome P450 11A1, Cytochrome P450 17A, Cytochrome P450 1A1, Cytochrome P450 1A2, Cytochrome P450 1 B1, Cytochrome P450 2A1, Cytochrome P450 2A3, Cytochrome P450 2A6, Cytochrome P450 2131, Cytochrome P450 21310, Cytochrome P450 2132, Cytochrome P450 2C11, Cytochrome P450 2C12, Cytochrome P450 2C19, Cytochrome P450 2C9, Cytochrome P450 2D6, Cytochrome P450 2E1, Cytochrome P450 2F2, Cytochrome P450 3A1, Cytochrome P450 3A4, Cytochrome P450 4A, Cytochrome P450 4A1, Damage specific DNA binding protein p48 subunit, Defender against cell death-1, Deleted in colorectal cancer, Deltalike protein, Dihydrofolate reductase, Disulfide isomerase related protein (ERp72), DNA binding protein inhibitor ID2, DNA dependent helicase, DNA dependent protein kinase, DNA ligase 1, DNA ligase IV, DNA mismatch repair protein (MLH1), DNA mismatch repair protein (PMS2), DNA mismatch repair/binding protein (MSH3), DNA polymerase alpha, DNA polymerase beta, DNA polymerase beta, DNA repair and recombination homologue (RAD 52), DNA repair helicase II ERCC-3, DNA repair protein (RAD 50), DNA repair protein (XRCC1), DNA repair protein XP-D, DNA replication factor C (36 kDa), DNA topoisomerase 1, DNA topoisomerase 11, Dopamine beta-hydroxylase, DRA, Dynein light chain 1, E2F, Early growth regulated protein 1, E-Cadherin, ECE-1 (endothelin converting enzyme), Endothelin-1, Enolase alpha, Enoyl CoA hydratase, Eotaxin, Epidermal growth factor, Epoxide hydrolase, ERA-B, ERCC 1 (excision repair protein), ERCC 3 (DNA repair helicase 11), ERCC 5 (excision repair protein), ERCC 6 (excision repair protein), ERK1, Erythropoietin, Erythropoietin receptor, ESelectin, Estrogen receptor, Farnesol receptor, Fas antigen, Fas associated death domain (FADD), Fas ligand, Fas/Apo1 receptor, Fatty acid synthase, Fatty acyl-CoA oxidase, Fatty acyl-CoA synthase, FEN-1 (endonuclease), Fibrinogen gamma chain, Fibronectin receptor, FIC1, Filagrin, Flavin containing monooxygenase 1, Flavin containing monooxygenase 3, FosB, Fra-1, Fucosyl transferase (alpha-1,2fucosyltransferase), Gadd153, Gadd45, Gamma-glutamyl hydrolase precursor, Gamma-glutamyl transpeptidase, GCLR, GCLS, Glucocorticoid receptor, Glucose-6-phosphate dehydrogenase, Glucose-regulated protein 170, Glucose-regulated protein 58, Glucose-regulated protein 78, Glucoseregulated protein 94, Glutamicoxaloacetic transaminase, Glutaminc-pyruvic transaminase, Glutathione peroxidase, Glutathione reductase, Glutathione S-transferase alpha subunit, Glutathione S-transferase 4a, Glutathione synthetase, Glyceraldehyde 3-phosphate dehydrogenase, GOS24 (zinc finger transcriptional regulator), Granulocyte-macrophage colony-stimulating factor, Growth-arrested-specific protein 1, Growth-arrested-specific protein 3, GT mismatch binding protein, H-cadherin, Heat shock protein 12, Heat shock protein 47, Heat shock protein 70, Heat shock protein 70.1, Heat shock protein 90, Helicase-like transcription factor, Heme binding protein 23, Heme oxygenase-1, Hepatic lipase, Hepatocyte growth factor, Hepatocyte growth factor activator, Hepatocyte growth factor receptor, Hepatocyte nuclear factor 4, Histone 2A, Histone 28, HMG CoA reductase, Hydroxyacyl CoA dehydrogenase, Hydroxysteroid sulfotransferase a, Hypoxanthine-guanine phosphoribosyltransferase, ICE-rel 11 (Caspase 4), ICH-2 cysteine protease=CASPASE 4, IkB-a, Insulin-like growth factor binding protein 1, Insulin-like growth factor binding protein 2, Insulin-like growth factor binding protien 3, Insulin-like growth factor I, Insulin-like growth factor 11, Integrin alpha, Integrin alpha L, Integrin betas, Integrin beta2, Intercellular adhesion molecule-1, Intercellular adhesion molecule-2, Intercellular adhesion molecule-3, Interferon gamma, Interferon inducible protein 10, Interferon inducible protein 15, Interleukin-1 alpha, Interleukin-12, Interleukin-2, Interleukin-4, Interleukin-5, Interleukin-6, Involucrin, JNK1 stress activated protein kinase, K-cadherin, Ki67, Lactate Dehydrogenase 8, Lactoferrin, Lipopolysaccharide binding protein, Lipoprotein lipase precursor, Liver fatty acid binding protein, L-myc, Low density lipoprotein receptor, Luteinizing hormone, Lysyl oxidase, Macrophage inflammatory protein-1 alpha, Macrophage inflammatory protein-1 beta, Macrophage inflammatory protein-2 alpha, Macrophage inflammatory protein-2 beta, Macrophage inflammatory protein-3 alpha, Macrophage inflammatory protein-3 beta, Malic enzyme, MAP kinase kinase, Matrix metal loproteinase1, Matrix metal loproteinase-2, MDM-2, MET proto-oncogene, Metallothionein 1, Metallothionein 2, Metallothionein 3, Metallothionein IA, Metallothionein IG, Metalregulatory transcription factor-1, Mitogen activated protein kinase (P38), Mitogen inducible gene (mig-2), MOAT-B (MRP/organic anion transporter), Monoamine oxidase A, Monoamine oxidase B, Multidrug resistance-associated protein, Multidrug resistant protein-1, Multidrug resistant protein-2, Multidrug resistant protein-3 =cMOAT2, MUTL homologue (MLH1), MutS Homologue (MSH2), Myeloid cell differentiation protein-1, Na/taurocholate cotransporting polypeptide, NADPH cytochrome P450 -oxidoreductase, NADPH cytochrome P450 reductase, NADPH quinone oxidoreductase-1 (DTDiaphorase), Natural killer cell-enhancing factor B, N-cadherin, NF-kappaB (p65), Nitric oxide synthase-1, inducible, Nucleoside diphosphate kinase beta isoform, 0-6-alkylguanine-DNAalkyltransferase, OBcadherin 1, OB-cadherin 2, Octamer binding protein 1, Octamer binding protein 2, Octamer binding protein 3, Oncostatin M, Organic anion transporter 1, Organic anion transporter 3, Organic anion transporter K1, Organic anion transporting polypeptide 1, Organic cation transporter 1, Organic cation transporter 2, Organic cation transporter 3, Organic cation transporter N1, Organic cation transporter N2, Ornithine decarboxylase, Osteopontin, Oxygen regulated protein 150, p53, PAPS synthetase, P-cadherin, PEGS (progression elevated gene 3), Peroxisomal 3-ketoacyl-CoA thiolase 1, Peroxisomal 3-ketoacylCoA thiolase 2, Peroxisomal acyl-CoA oxidase, Peroxisomal fatty acyl-CoA oxidase, Peroxisome assembly factor 1, Peroxisome assembly factor 2, Peroxisome biogenesis disorder protein-1, Peroxisome biogenesis disorder protein-11, Peroxisome biogenesis disorder protein-4, Peroxisome hydratase, Peroxisome proliferator activated receptor alpha, Peroxisome proliferator activated receptor gamma, Phenol sulfotransferase, Phosphoenolpyruvate carboxykinase, Phosphoglyceride kinase, Phospholipase A2, Plasminogen activator inhibitor 2, Platelet derived growth factor B, Platelet/endothelial cell adhesion molecule-1, Poly (ADP ribose) polymerase, Proliferating cell nuclear antigen gene, Prostaglandin H synthase, Protein kinase C betal, Protein-tyrosine phosphatase, Putative protein tyrosine phosphatase, RAID, RAID 51 homologue, RANTES, Ref 1, Replication factor C, 40-kDa subunit (Al), Replication protein A (70 kDa subunit), Retinoblastoma, Retinoblastoma related protein (P 107), Retinoid X receptor alpha, Retinoid X receptor beta, Retinoid X receptor gamma, Ribonucleotide reductase M1 subunit, Ribosomal protein L13A, Ribosomal protein S9, RNA-dependent helicase, ROAT1 (renal organic anion transporter), Serum amyloid A1, Serum amyloid A2alpha, Sister of p-glycoprotein, Sodium/bile acid cotransporter, Sonic hedgehog gene, SQM1, Superoxide Dismutase Cu/Zn, Superoxide dismutase Mn, T-cell cyclophilin, Tenascin, Thiopurine methyltransferase, Thioredoxin, Thrombospondin 2, Thymidine kinase, Thymidylate synthase, Thymosin beta-10, Tissue inhibitor of metalloproteinases-1, Tissue transglutaminase, Transcription factor IID, Transferrin, Transforming growth factor-beta 3, Tumor necrosis factor associated factor 2 (TRAF2), Tumor necrosis factor receptor 1, Tumor necrosis factor receptor 2, Tumor necrosis factor receptor-1 associated protein (TRADD), Tumor necrosis factor-alpha, Tumor necrosis factorbeta, Type 1 interstitial collagenase, Tyrosine aminotransferase, Tyrosine protein kinase receptor (UFO), Ubiquitin, Ubiquitin conjugating enzyme (Rad 6 homologue), Ubiquitin-homology domain protein PIC1, UDPglucuronosyltransferase 1, UDP-glucuronosyltransferase 1A6, UDPglucuronosyltransferase 2, UDP-glucuronosyltransferase 28, Uncoupling protein 1, Uncoupling protein 2, Uncoupling protein 3, Urate oxidase, UV excision repair protein RAD 23 (XP-C), Vascular cell adhesion molecule 1 (VCAM-1), Vascular endothelial growth factor, Vascular endothelial growth factor D, Very long-chain acyl-CoA dehydrogenase, Vimentin, Vitellogenin, Waf1, XRCC1 (DNA repair protein).

Also, the invention particularly embraces the incorporation of antisense oligonucleotides that inhibit the expression of a target gene, e.g. an oncogene.

As noted, modified vectors according to the invention have application for use in gene or cell therapy strategies, vaccines and for assessing the function of target genes which are expressed on the vector. These applications are discussed further below.

Vaccination Strategies

In this embodiment of the invention, a vector, e.g. a virus or bacterium, is constructed wherein an antigen, typically an immunodominant epitope such as a viral capsid protein is placed under the regulatory control of a stress inducible promoter. This is effected by replacing the endogenous antigen gene promoter with a stress-inducible promoter, e.g. HSP70B or HSP70.

Thereupon, the vector is introduced via well known means of administration of viral or bacterial vaccines, e.g. injection (intramuscular, intravenous, subcutaneous), oral, intravaginal, transdermal, etc., comprised in a pharmaceutically acceptable carrier. After administration, the vaccinated subject is then exposed to stress induction conditions, e.g. by application of thermal stress. Thereupon, the vector expresses the particular antigen, e.g. a viral or bacterial protein. The host is then assessed to determine whether immunization has been achieved, e.g. by determining whether antibodies have been produced against the particular antigen. If the requisite degree of immunization has not been realized, the subject is again exposed to the stress stimuli.

The advantage of this methodology is that it may avoid the need for multiple “booster injections”. Essentially, the subject after administration has a latent booster supply of antigen which can be “turned on” as desired merely by expressing the subject to stress stimuli thereby inducing the expression of the antigen. This is advantageous from cost, safety and convenience issues.

Second, it would better avoid the well known risks of current live attenuated vaccines, such as polio vaccine. In some very susceptible individuals, even the attenuated replication or expression of pathogenic traits can still cause serious disease. Because replication and/or pathogenic expression of the vectors comprising this invention are not merely attenuated, but well controlled by exogenous inducers, such replication or expression can be terminated or reduced in the event of an idiosyncratic response by removing the stimulus or at least more consistently regulated than reliance on attenuation. Moreover, because replication or expression can be controlled spatially (e.g., by heating a target tissue), it will be far easier to avoid side effects caused by the vaccine replicating or expressing in other tissues, such as the brain. Third, it enables the more efficient construction of such vaccines. Because replication and/or expression can be more easily established through ordinary genetic engineering methods, than attempting to “develop” attenuate strains by the means currently employed. Furthermore, because the vectors comprising this invention are only modified to the extent necessary to establish the desired control, the resulting vaccine will have more predictable and robust qualities than common attenuated strains that usually have multiple genetic modifications throughout their genome.

Pathogenicity Strategies

In this embodiment of the invention, a vector, e.g. a virus, plasmid or another bacterium is engineered such that a gene which is involved in the pathogenicity, immunogenicity or replication of a particular vector, e.g. a toxin, is placed under the regulatory control of a stress regulatory sequence.

In this strategy, the vector will be administered to a subject in need of such administration. For example, this approach can be utilized to selectively induce expression of the vector at a desired target, e.g. tumor site, infection or inflammation site, etc. In this embodiment, the vector may ideally comprise a gene encoding a desired enzyme, ligand, epitope, or therapeutic protein. Accordingly, by this strategy, when the vector is exposed to stress inducible conditions at a particular site, e.g. a tumor or infection or inflammation site, it results in the selective delivery of the desired gene product at the target site. For example, this strategy can be used to deliver an enzyme that converts a prodrug to an active form, a cytokine, a ligand that inhibits expression of a tumor-associated antigen, as well as other appropriate therapeutic agents. The selection of the particular agent depends upon the nature of the disease condition and the target site.

This approach is advantageous as it should eliminate non-specific toxicity. Essentially, the active agent will only be delivered to target site (e.g. tumor) exposed to thermal stress, thereby minimizing the potential for the active agent to kill non-target cells. Also, this approach should reduce the requisite dosage as administered active agent that the subject is exposed to. This may further reduce the likelihood of the subject eliciting an unwanted response against the particular agent.

Tissue Ablation or Modification

This strategy is similar to the previous strategy. Essentially, in this embodiment, a vector is constructed, e.g. a plasmid having a gene that encodes a polypeptide that is itself cytotoxic or which encodes a polypeptide that results in the production of a cytotoxic moiety (e.g. converts a pro-toxin to a cytotoxin, or complexes to form a cytotoxic moiety), which is expressed under the regulatory control of a stress gene regulatory element.

The vector is then administered, e.g. by injection into a tumor site, and then induced to provide the necessary effect, such as cell lysis. There are several advantages to this approach. First, by providing for highly efficient spatial and temporal control of such vectors, the physician can be selective about the level of killing permitted. This is highly useful where there is a desire to preserve nearby features (e.g. killing of metastases close to critical organs or nerves); or to modulate the quantity of killing so that it can be modulated on a patient or cell specific basis (e.g., leukoreduction in response to leukaemic blast crisis or acute graft v. host disease). Second, more importantly, it provides clear improvement over the prior art in that the ability to selectively kill or modify cells is not tied to the nature or biology of the cells (e.g. particular cell types or diseases, or genetic background of the target cells), as in the case with replication controlled vectors controlled by, for example, endogenous p53 expression or drug induction.

### Functional Gene Assays

Gene Therapy

Cancer cells escape normal growth control mechanisms as a consequence of activating mutations and/or increased expression of one or more cellular protoncogenes and/or inactivating mutations and/or decreased expression of one or more tumor suppressor genes (e.g., p53, RD, DCC, NF-1 . . . ). Most oncogene and tumor suppressor gene products are components of signal transduction pathways that control cell cycle entry or exit, promote differentiation, sense DNA damage and initiate repair mechanisms, and/or regulate cell death programs.

Some oncogenes have been found to possess characteristic activating mutations in a significant fraction of certain cancers. For example, particular mutations in the ras^{H} and the ras^{K} coding regions (Parada et al. (1984) Nature 312:649) and the APC gene (Powell et al. (1992) Nature 359:235) are associated with oncogenic transformation of cultured cells and are present in a striking percentage of specific human cancers (e.g. colon adenocarcinoma, bladder carcinoma, lung carcinoma, adenocarcinoma and hepatocarcinoma). These findings have led to the development of diagnostics and therapeutic reagents that specifically recognize the activated form(s) of such oncogenes (U.S. Pat. Nos. 4,798,787 and 4,762,706).

Recent approaches for performing gene therapy to correct or supplement defective alleles which cause congenital diseases have been attempted with reports of limited initial success. Some gene therapy approaches involve transducing a polynucleotide sequence capable of expressing a functional copy of a defective allele into a cell in vivo using replication-deficient recombinant adenovirus (Rosenfield et al. (1992) Cell 68:143). Some of these gene therapy methods are efficient at transducing polynucleotides into isolated cells explanted from a patient, but have not been shown to be highly efficient in vivo.

In the past decade, adenovirus vectors have generated tremendous interest in gene therapy applications. Nevertheless, the efficiency of gene therapy strategies using those adenoviruses is limited by the poor and non-specific distribution of the viral vectors in the malignant tissue. To solve these problems, the authors of this invention have developed a new generation of tumor-specific, conditionally replicative adenoviruses.

The use of adenoviral vectors in gene therapy has a number of advantages. The wild type viruses are able to replicate in both quiescent and dividing cells. Their lack of tropism allows them to infect multiple cell types. Further, their relatively small genome allows convenient genetic constructions to be made, while the deletion vectors allow sufficient space to install multiple gene constructs.

Depending on the application, the fact that adenoviral vectors do not integrate into cellular genome, can be advantageous. On the negative side, particularly with the vectors derived from the Adenovirus 5, a large part of the human population has antibodies against this common virus.

Adenovirus has five transcription units that are expressed before the onset of viral replication, called early units (E1a, E1b, E2, E3 and E4). Most adenoviral vectors used in gene therapy to date have employed replication-defective adenoviruses, generally lacking the E1 a region and/or the E3 region, and are referred to as gutted adenoviruses. These vectors allow the infection of the targeted tissue, expression of the transgene, but not the dissemination to other tissues and organs through viral replication.

One system of using a control of adenoviral vector replication has been described: U.S. Pat. No. 5,677,178 describes a mutant vector, deleted in the E1b region, that only allows its replication in the absence of functional p53. U.S. Pat. No. 5,801,0298 claims the use of specific adenoviral mutants that are deleted in the El a region and that kills cells deficient in the functional RB activity. U.S. Pat. No. 5,846,945 claims the use of combinations of the above-mentioned mutant adenoviral vector (U.S. Pat. No. 5,677,178) with chemotherapeutic agents.

The above examples are carried out using the human promoter HSP70B which is advantageous, relative to other HSP promoters as it has been demonstrated to have essentially no basal expression, very high expression during induction and ready reversibility when the induction stimulus is removed.

Further, essentially any gene essential for adenoviral replication can be employed in this invention. Such genes include, but are not limited to, genes from the E1a and E1b regions of adenoviral genomes. This invention can be employed with any adenovirus serotype, or any genetically modified variant. Further, this invention is equally applicable to other lytic viruses, including but not limited to members of the herpes virus family, as well as non-lytic viruses, microbes and vectors.

This subject vectors can be optionally combined with the inclusion of therapeutic genes such as those encoding apoptosis related proteins, those inhibiting cell cycle, those inhibiting or inducing angiogenesis, tumor suppressors as well as those genes mediating cell death via the action of a pro-drug. Essentially, any gene or combination of genes may be introduced into adenoviral vectors using this invention.

Moreover, the use of human HSP70 promoter has numerous advantages. It has been shown that some tumor suppressors, like for example p53, can repress transcription from this promoter (Agoff et al. (1993) Science 259). Consequently, adenovirus infecting non-cancer cells would not replicate.

Adenoviral vectors as described in this invention are one example of the present invention. Clearly, any vector that can be advantageously employed with such a replication control system is included in this invention. Wherever a gene that controls replication of such vectors is placed under the control of a stress-inducible promoter, the teachings of this invention can be applied.

In Vivo Administration

Alternatively, the vector of the present invention can also be used for the in vivo gene transfer, using methods which are known to those of skill in the art. The insertion of genes into cells for the purpose of medicinal therapy is a rapidly growing field in medicine which has enormous clinical potential. Research in gene therapy has been on-going for several years, and has entered human clinical trials. Zhu, et al., (1993) Science 261: 209-211, incorporated herein by reference, describes the intravenous delivery of cytomegalovirus (CMV)-chloramphenicol acetyltransferase (CAT) expression plasmid using DOTMA-DOPE complexes. Hyde, et al., (1993) Nature 362: 250-256, incorporated herein by reference, describes the delivery of the cystic fibrosis transmembrane conductance regulator (CFTR) gene to epithelia of the airway and to alveoli in the lung of mice, using liposomes. Brigham, et al.,(1989) Am. J Med. Sci. 298: 278-281, incorporated herein by reference, describes the in vivo transfection of lungs of mice with a functioning prokaryotic gene encoding the intracellular enzyme chloramphenicol acetyltransferase (CAT).

For in vivo administration, the pharmaceutical compositions are preferably administered parenterally, i.e., intraarticularly, intravenously, intraperitoneally, subcutaneously, or intramuscularly. More preferably, the pharmaceutical compositions are administered locally, e.g. by IV catheter subcutaneously such that it is delivered at a target site. Alternatively, the vector can be administered. For example, see Stadler, et al., U.S. Pat. No. 5,286,634, which is incorporated herein by reference. Intracellular nucleic acid delivery has also been discussed in Straubringer, et al., (1983) Methods In Enzymology, Academic Press, New York. 101:512-527; Mannino, et al., (1988) Biotechniques 6:682-690; Nicolau, et al., (1989) Crit. Rev. Ther. Drug Carrier Syst. 6:239-271, and Behr, (1993) Acc. Chem. Res. 26:274-278. Still other methods of administering therapeutics are described in, for example, Rahman et al., U.S. Pat. No. 3,993,754; Sears, U.S. Pat. No. 4,145,410; Papahadjopoulos et al., U.S. Pat. No. 4,235,871; Schneider, U.S. Pat. No. 4,224,179; Lenk et al., U.S. Pat. No. 4,522,803; and Fountain et al., U.S. Pat. No. 4,588,578.

In certain embodiments, the pharmaceutical preparations may be contacted with the target tissue by direct application of the preparation to the tissue. The application may be made by topical, “open” or “closed” procedures. By “topical”, it is meant the direct application of the pharmaceutical preparation to a tissue exposed to the environment, such as the skin, oropharynx, external auditory canal, and the like. “Open” procedures are those procedures which include incising the skin of a patient and directly visualizing the underlying tissue to which the pharmaceutical preparations are applied. This is generally accomplished by a surgical procedure, such as a thoracotomy to access the lungs, abdominal laparotomy to access abdominal viscera, or other direct surgical approach to the target tissue. “Closed” procedures are invasive procedures in which the internal target tissues are not directly visualized, but accessed via inserting instruments through small wounds in the skin. For example, the preparations may be administered to the peritoneum by needle lavage. Likewise, the pharmaceutical preparations may be administered to the meninges or spinal cord by infusion during a lumbar puncture followed by appropriate positioning of the patient as commonly practiced for spinal anesthesia or metrazamide imaging of the spinal cord. Alternatively, the preparations may be administered through endoscopic devices.

The subject vectors can also be administered in an aerosol inhaled into the lungs (see, Brigham, et al., (1989) Am. J Sci. 298(4):278-281 or by direct injection at the site of disease (Culver, (1994) Human Gene Therapy, MaryAnn Liebert, Inc., Publishers, New York. pp.70-71).

The methods of the present invention may be practiced in a variety of hosts. Preferred hosts include mammalian species, such as humans, non-human primates, dogs, cats, cattle, horses, sheep, and the like.

The amount of vector administered will depend upon the particular vector used, the disease state being diagnosed; the age, weight, and condition of the patient and the judgement of the clinician; but will generally be between about 0.01 and about 50 mg per kilogram of body weight; preferably between about 0.1 and about 5 mg/kg of body weight or about 10⁸-10¹⁰ particles per injection.

Formulations suitable for administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations of packaged nucleic acid can be presented in unit-dose or multi-dose sealed containers, such as ampules and vials. Injection solutions and suspensions can be prepared from sterile powders, granules, and tablets of the kind previously described.

A vector dose which is sufficient to effect a beneficial therapeutic response in the patient over time, or to inhibit infection by a pathogen, is administered to a patient. A therapeutically effective dose is an amount sufficient to cure, or at least partially arrest, the symptoms of the disease and its complications. Effective doses of the compositions of the present invention, for the treatment of the above described conditions will vary depending upon many different factors, including means of administration, target site, physiological state of the patient, and other medicants administered. Thus, treatment dosages will need to be titrated to optimize safety and efficacy. In determining the effective amount of the vector to be administered, the physician evaluates the particular nucleic acid used, the disease state being diagnosed; the age, weight, and condition of the patient, circulating plasma levels, vector toxicities, progression of the disease, and the production of anti-vector antibodies. The size of the dose also will be determined by the existence, nature, and extent of any adverse side-effects that accompany the administration of a particular vector. Doses ranging from about 10 ng to 1 g, 100 ng to 100 mg, 1 μg to 10 mg, or 30-300 μg DNA per patient are typical. Doses generally range between about 0.01 and about 50 mg per kilogram of body weight; preferably between about 0.1 and about 5 mg/kg of body weight or about 10⁸-10¹⁰ particles per injection. In general, the dose equivalent of a naked nucleic acid from a vector is from about 1 μg to 100 μg for a typical 70 kilogram patient, and doses of vectors which include a retroviral particle are calculated to yield an equivalent amount of inhibitor nucleic acid.

Prior to infusion, blood samples are obtained and saved for analysis. Between 10⁸ and 1×10¹² vectors are infused intravenously over 60-200 minutes. Vital signs and oxygen saturation by pulse oximetry are closely monitored. Blood samples are obtained 5 minutes and 1 hour following infusion and saved for subsequent analysis. At the physicians discretion, reinfusion is repeated every 2 to 3 months for a total of 4 to 6 treatments in a one year period. After the first treatment, infusions can be performed on a outpatient basis at the discretion of the clinician. If the reinfusion is given as an outpatient, the participant is monitored for at least 4, and preferably 8 hours following the therapy.

If a patient undergoing infusion of a vector or transduced cell develops fevers, chills, or muscle aches, he/she receives the appropriate dose of aspirin, ibuprofen or acetaminophen. Patients who experience reactions to the infusion such as fever, muscle aches, and chills are premedicated 30 minutes prior to the future infusions with either aspirin, acetaminophen, or diphenhydramine. Meperidine is used for more severe chills and muscle aches that do not quickly respond to antipyretics and antihistamines. Vector infusion is slowed or discontinued depending upon the severity of the reaction.

Diseases Treatable According to Invention

Some methods of gene therapy serve to compensate for a defect in an endogenous gene by integrating a functional copy of the gene into the host chromosome. The inserted gene replicates with the host DNA and is expressed at a level to compensate for the defective gene. Diseases amenable to treatment by this approach are often characterized by recessive mutations. That is, both copies of an endogenous gene must be defective for symptoms to appear. Such diseases include, for example, cystic fibrosis, sickle cell anemia, thalassemia, phenylketonuria, galactosemia, Wilson's disease, hemochromatosis, severe combined immunodeficiency disease, alpha-1-antitrypsin deficiency, albinism, alkaptonuria, lysosomal storage diseases, Ehlers-Danlos syndrome, hemophilia, glucose-6-phosphate dehydrogenase deficiency, alphagammaglobulimenia, diabetes insipidus, Leech-Nyhan syndrome, muscular dystrophy, Wiskott-Aldrich syndrome, Fabry's disease, fragile X-syndrome, and the like. Other recessive mutations are known in the art, and the use of the methods of the present invention to treat them is contemplated herein.

There are several methods for introducing an exogenous functional gene to compensate for the above genetic defects. In one approach, cells are removed from a patient suffering from the disease and contacted with a vector in vitro. Cells should be removed from a tissue type in which disease symptoms are manifested. If the cells are capable of replication, and the vector used includes a selective marker, cells having internalized and expressed the marker can be selected. Particularly if selection is not performed, it is important that the frequency of gene transfer into cells be high, for example, at least about 1, 5, 10, 25 or 50% of cells.

After integration of the vector into the cellular genome, and optionally, selection, cells are reintroduced into the patient. In this application, and others discussed below (except site-specific recombination to correct dominant mutations), it is not necessary that the gene supplied be delivered to the same site as is occupied by the defective gene for which it is compensating.

Alternatively, vectors can be introduced directly into a patient as a pharmaceutical composition. The complex is delivered to the tissue(s) affected by the genetic disorder being treated in a therapeutically effective dose. In this and other methods, a therapeutically effective dose is an amount sufficient to cure, or at least partially arrest, the symptoms of the disease and its complications. Effective doses of the compositions of the present invention, for the treatment of the above described conditions will vary depending upon many different factors, including means of administration, target site, physiological state of the patient, and other medicants administered. Thus, treatment dosages will need to be titrated to optimize safety and efficacy. Doses ranging from about 10 ng to 1 g, 100 ng to 100 mg, 1 μg to 10 mg, or 30-300 μg DNA per patient are typical. Routes of administration include oral, nasal, gastric, intravenous, intradermal and intramuscular.

i. Stem Cell Therapy

The subject vector, e.g., recombinant viruses and plasmids can also be used to transfect embryonic stem cells or zygotes to achieve germline alterations. See Jaenisch, (1988) Science, 240:468-1474; Gordon et al. (1984) Methods Enzymol. 101:414; Hogan et al., (1986) Manipulation of the Mouse Embryo: A Laboratory Manual, C.S.H.L. N.Y.; and Hammer et al. (1985) Nature 315:680; Gandolfi et al. (1987) J. Reprod. Pert. 81:23-28; Rexroad et al. (1988) J. Anim. Sci. 66:947-953 and Eyestone et al. (1989) J. Reprod. Pert. 85:715-720; Camous et al. (1984) J. Reprod. Pert. 72:779-785; Heyman et al. (1987) Theriogenology 27:5968. However, these methods are presently more suitable for veterinary applications that human treatment due to ethical and regulatory constraints in manipulating human embryos.

As an example, cystic fibrosis (CF) is a usually fatal recessive genetic disease, having a high incidence in Caucasian populations. The gene responsible for this disease was isolated by Riordan et al, (1989) Science 245:1059-1065. It encodes a protein called the cystic fibrosis transmembrane conductance regulator (CFTR) which is involved in the transfer of chloride ions (C1⁻) through epithelial cell membranes. Mutations in the gene cause defects of C1⁻ secretion in epithelial cells leading to the various clinical manifestations. Although CF has a number of symptoms including thickened exocrine gland secretions, pancreatic deficiency, intestinal blockage and malabsorption of fat, the most serious factor affecting mortality is chronic lung disease. Accordingly, to treat a CF patient, a vector containing a coding sequence for a functional CFTR gene product can be introduced into the patient via nasal administration so that the nucleic acid composition reaches the lungs. The dose of vector is preferably about 10⁸-10¹⁰ particles.

As another example, defects in the alpha or beta globin genes (see McDonagh & Nienhuis in Hematology of Infancy and Childhood (eds. Nathan & Oski, Saunders, Pa., 1992) at pp. 783-879) can be compensated for by ex vivo treatment of hemopoietic stem cells with a nucleic acid containing a functional copy of the gene. The gene integrates into the stem cells which are then reintroduced into the patient. Defects in the gene responsible for Fanconi Anemia Complement Group C can be treated by an analogous strategy (see Walsh et al., (1994) J. Clin. Invest. 94:1440-1448).

ii. Cancer Therapy

Other applications of the subject vectors include the introduction of a functional copy of a tumor suppressor gene into cancerous cell or cells at risk of becoming cancerous. D. Pardoll, (1992) “Immunotherapy with cytokine gene-transduced tumor cells: the next wave in gene therapy for cancer”, Curr. Opin. Oncol. 4:1124-1129; Uckert and Walther, (1994) “Retrovirus-mediated gene transfer in cancer therapy”, Pharmac. Ther. 63:323-347; Individuals having defects in one or both copies of an endogenous tumor suppressor gene are particularly at risk of developing cancers. For example, Li-Fraumeni syndrome is a hereditary condition in which individuals receive mutant p53 alleles, resulting in the early onset of various cancers (Harris, (1993) Science 262:1980-1981, Frebourg et al., (1992) PNAS 89:6413-6417; Malkin et al., (1990) Science 250:1233). Expression of a tumor suppressor gene in a cancerous cell or a cell at risk of becoming cancerous is effective to prevent, arrest and/or reverse cellular proliferation and other manifestations of the cancerous state. Suitable tumor suppressor genes for use in the invention include p53 (Buchman et al., (1988) Gene 70:245-252), APC, DCC, Rb, WT1, and NF1 (Marx, (1993) Science 260:751-752; Marshall, (1991) Cell 64:313-326). Vectors bearing a functional copy of a tumor suppressor gene are usually administered in vivo by the route most proximal to the intended site of action. For example, skin cancers can be treated by topical administration and leukemia by intravenous administration. The methods of the invention are useful for treating a wide variety of cancers, among them prostate, glyoma, ovarian, and mammary tumors.

Suppression of Gene Expression

Methods of gene therapy using the nucleic acid constructs of the invention can also be used for prophylactic or therapeutic treatment of patients or cells, infected with or at risk of being infected with, a pathogenic microorganism, such as HIV. The effectiveness of antisense molecules in blocking target gene functions has been demonstrated in a number of different systems (Friedman et al. (1988), Nature 335:452-54, Malim et al., (1989) Cell 58:205-14 and Trono et al., (1989) Cell 59:11320). The vector used includes a DNA segment encoding an antisense transcript, which is complementary to a segment of the gene. Where the gene is from a pathogenic microorganism, it should preferably play an essential role in the life cycle of the and should also be unique to the microorganism (or at least absent from the genome of the patient undergoing therapy). For example, suitable sites for inhibition on the HIV virus includes TAR, REV or nef (Chatterjee et al., (1992) Science 258:1485-1488). Rev is a regulatory RNA binding protein that facilitates the export of unspliced HIV pre mRNA from the nucleus. Malim et al., (1989) Nature 338:254. Tat is thought to be a transcriptional activator that functions by binding a recognition sequence in 5′ flanking mRNA. Karn & Graeble, (1992) Trends Genet. 8:365. The vector is introduced into leukocytes or hemopoietic stem cells, either ex vivo or by intravenous injection in a therapeutically effective dose. The treatment can be administered prophylactically to HIVpersons, or to persons already infected with HIV.

Cells to be Transformed

The vectors and methods of the present invention are used to transfer genes into a wide variety of cell types, in vivo and in vitro. Among those most often targeted for gene therapy are precursor (stem) cells, especially hematopoietic stem cells. Other cells include those of which a proportion of the targeted cells are nondividing or slow dividing. These include, for example, fibroblasts, keratinocytes, endothelial cells, skeletal and smooth muscle cells, osteoblasts, neurons, quiescent lymphocytes, terminally differentiated cells, slow or noncycling primary cells, parenchymal cells, lymphoid cells, epithelial cells, bone cells, etc. The methods and compositions can be employed with cells of a wide variety of vertebrates, including mammals, and especially those of veterinary importance, e.g, canine, feline, equine, bovine, ovine, caprine, rodent, lagomorph, swine, etc., in addition to human cell populations.

To the extent that tissue culture of cells may be required, it is well known in the art. Freshney (1994) (Culture of Animal Cells, a Manual of Basic Technique, third edition Wiley-Liss, New York); Kuchler et al. (1977) Biochemical Methods in Cell Culture and Virology, Kuchler, R. J., Dowden, Hutchinson and Ross, Inc., and the references cited therein provides a general guide to the culture of cells: Cultured cell systems often will be in the form of monolayers of cells, although cell suspensions are also used.

Gene therapy relies on the efficient delivery of therapeutic genes to target cells. Most of the somatic cells that have been targeted for gene therapy, e.g., hematopoietic cells, skin fibroblasts and keratinocytes, hepatocytes, endothelial cells, muscle cells and lymphocytes, are normally non-dividing. Retroviral vectors, which are the most widely used vectors for gene therapy, unfortunately require cell division for effective transduction (Miller et al., (1990) Mol. Cell. Biol. 10:4239-4242). This is also true with other gene therapy vectors such as the adeno-associated vectors (Russell et al., (1991) Proc. Natl. Acad. Sci. USA 91:8915-8919; Alexander et al., (1994) J. Virol. 68:8282-8287; Srivastrava, (1994) Blood Cells 20:531-538). Recently, HIV-based vectors has been reported to transfect non-dividing cells. Nonetheless, the majority of stem cells, a preferred target for many gene therapy treatments, are normally not proliferating. Thus, the efficiency of transduction is often relatively low. and the gene product may not be expressed in therapeutically or prophylactically effective amounts. This has led investigators to develop techniques such as stimulating the stem cells to proliferate prior to or during gene transfer (e.g., by treatment with growth factors) Pretreatment with 5-fluorouracil, infection in the presence of cytokines, and extending the vector infection period to increase the likelihood that stem cells are dividing during infection, but these have met with limited success.

After a vector that optionally encodes a gene of interest wherein the replicative pathogenicity and/or immunogenicity treatment is under the control of a hsp promoter is administered, it is important to detect which cells or cell lines express the gene product and to assess the level of expression of the gene product in engineered cells. This requires the detection of nucleic acids that encode the gene products.

Nucleic acids and proteins are detected and quantified herein by any of a number of means well known to those of skill in the art. These include analytic biochemical methods such as spectrophotometry, radiography, electrophoresis, capillary electrophoresis, high performance liquid chromatography (HPLC), thin layer chromatography (TLC), hyperdiffusion chromatography, and the like, and various immunological methods such as fluid or gel precipitin reactions, immunodiffusion (single or double), immunoelectrophoresis, radioimmunoassays (RIAs), enzymelinked imrnunosorbent assays (ELISAs), immunofluorescent assays, and the like. The detection of nucleic acids proceeds by well known methods such as Southern analysis, northern analysis, gel electrophoresis, PCR, radiolabeling, scintillation counting, and affinity chromatography.

The selection of a nucleic acid hybridization format is not critical. A variety of nucleic acid hybridization formats are known to those skilled in the art. For example, common formats include sandwich assays and competition or displacement assays. Hybridization techniques are generally described in Nucleic Acid Hybridization, A Practical Approach, Ed. Hames, B. D. and Higgins, S. J., IRL Press, 1985.

The sensitivity of the hybridization assays may be enhanced through use of a nucleic acid amplification system which multiplies the target nucleic acid being detected. In vitro amplification techniques suitable for amplifying sequences for use as molecular probes or for generating nucleic acid fragments for subsequent subcloning are known. Examples of techniques sufficient to direct persons of skill through such in vitro amplification methods, including the polymerase chain reaction (PCR) the ligase chain reaction (LCR), Qβ-replicase amplification and other RNA polymerase mediated techniques (e.g., NASBA) are found in Berger, Sambrook, and Ausubel, as well as Mullis et al. (1987), U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990), C & EN 36-47; The Journal Of NIH Research (1991), 3: 81-94; (Kwoh et al. (1989), Proc. Natl. Acad. Sci. USA, 86:1173; Guatelli et al. (1990), Proc. Natl. Acad. Sci. USA, 87:1874; Lomell et al. (1989), J. Clin. Chem., 35:1826; Landegren et al. (1988), Science, 241:1077-1080; Van Brunt (1990), Biotechnology, 8:291-294; Wu and Wallace (1989), Gene, 4:560; Barringer et al. (1990), Gene, 89:117, and Sooknanan and Malek (1995), Biotechnology, 13:563-564. Improved methods of cloning in vitro amplified nucleic acids are described in Wallace et al., U.S. Pat. No. 5,426,039. Other methods recently described in the art are the nucleic acid sequence based amplification (NASBA™, Cangene, Mississauga, Ontario) and Q Beta Replicase systems. These systems can be used to directly identify mutants where the PCR or LCR primers are designed to be extended or ligated only when a select sequence is present. Alternatively, the select sequences can be generally amplified using, for example, nonspecific PCR primers and the amplified target region later probed for a specific sequence indicative of a mutation.

Oligonucleotides for use as probes, e.g., in in vitro amplification methods, for use as gene probes, or as inhibitor components are typically synthesized chemically according to the solid phase phosphoramidite triester method described by Beaucage and Caruthers (1981), Tetrahedron Letts., 22(20):1859-1862, e.g., using an automated synthesizer, as described in Needham-Van Devanter et al. (1984), Nucleic Acids Res., 12:6159-6168. Purification of oligonucleotides, where necessary, is typically performed by either native acrylamide gel electrophoresis or by anion-exchange HPLC as described in Pearson and Regnier (1983), J. Chrom., 255:137-149. The sequence of the synthetic oligonucleotides can be verified using the chemical degradation method of Maxam and Gilbert (1980) in Grossman and Moldave (eds.) Academic Press, New York, Methods in Enzymology, 65:499-560.

An alternative means for determining the level of expression of the vector is in situ hybridization. in situ hybridization assays are well known and are generally described in Angerer et al. (1987), Methods Enzymol., 152:649660. In an in situ hybridization assay cells are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labelled with radioisotopes or fluorescent reporters.

Detection of Foreign Gene Products

The expression of the vector containing a replication, pathogenicity or immunogenicity gene of interest under the control of a hsp promoter to produce a product may be detected or quantified by a variety of methods. Preferred methods involve the use of specific antibodies.

Methods of producing polyclonal and monoclonal antibodies are known to those of skill in the art. See, e.g., Coligan (1991), Current Protocols In Immrtolooy, Wiley/Greene, NY; and Harlow and Lane (1989), Antibodies: A Laboratory Manual, Cold Spring Harbor Press, NY; Stites et al. (eds.) Basic And Clinical Immology (4th ed.) Lange Medical Publications, Los Altos, Calif., and references cited therein; Goding (1986), Monoclonal Antibodies: Principles And Practice (2d ed.) Academic Press, New York, N.Y.; and Kohler and Milstein (1975), Nature, 256:495-497. Such techniques include antibody preparation by selection of antibodies from libraries of recombinant antibodies in phage or similar vectors. See, Huse et al. (1989), Science, 246:1275-1281; and Ward et al. (1989), Nature, 341:544-546. Specific monoclonal and polyclonal antibodies and antisera will usually bind with a KD of at least about 0.1 mM, more usually at least about 1 μM, preferably at least about 0.1 μM or better, and most preferably, 0.01 μM or better.

The presence of a desired polypeptide (including peptide, transcript, or enzymatic digestion product) in a sample may be detected and quantified using Western blot analysis. The technique generally comprises separating sample products by gel electrophoresis on the basis of molecular weight, transferring the separated proteins to a suitable solid support, (such as a nitrocellulose filter, a nylon filter, or derivatized nylon filter), and incubating the sample with labeling antibodies that specifically bind to the analyte protein. The labeling antibodies specifically bind to analyte on the solid support. These antibodies are directly labeled, or alternatively are subsequently detected using labeling agents such as antibodies (e.g., labeled sheep antimouse antibodies where the antibody to an analyte is a murine antibody) that specifically bind to the labeling antibody.

Heat Induction of the bsp Promoter

One aspect of the present invention is the ability to selectively activate stress-inducible, e.g. heat-inducible, promoters, e.g. using localized heat. In particular, it is important to stress inducible, e.g. controllably heat cells within a target area located within deep tissue while minimizing heating of surrounding cells.

Localized heating of deep lying tissues can be accomplished by invasive or noninvasive methods (without opening the skin). Among the invasive methods, the introduction of a catheter with a heated tip can be used. Alternatively, a catheter with an optical guide can be used. A laser beam can then be directed through the catheter to the targeted tissue and heat can be deposited using direct radiation (for example using infrared light). Although irradiation by laser has been proposed for heating deep tissue, its use in medicine has been limited by optical absorption and thermal diffusion.

In the preferred embodiment, local heating is achieved by noninvasive means. FIG. 1, taken from Ernst et al., Principles Of Nuclear Radiation In One And Two Dimensions, Oxford University Press, 1987, illustrates the attenuation of electromagnetic and ultrasound radiation. For heating of deep lying tissues, a transition area in FIG. 1 is used (since both absorption and penetration are needed). The X-ray region of the spectrum uses ionizing radiation which is hazardous. The radio frequency region has a wavelength of more than 10 cm. Acoustical radiation is strongly absorbed for wavelengths below 1 mm. Since the ability to focus is limited to approximately half the wavelength, a focus diameter of 5 cm or more can be attained by radio frequency. This is generally not localized enough to treat small lesions. Ultrasound can be applied with a short enough wavelength to be localized and can penetrate deeply and is to some extent absorbed by body tissues. Therefore, the preferred method for noninvasive local heating is focused ultrasound.

It is known that ultrasound can be aimed at a defined target area, and that prolonged exposure of living tissues to ultrasound can raise the temperature of the exposed tissue. In particular focused ultrasound has been known to be very effective to locally heat tissue so long as there is an acoustic path from the surface to the lesion free of air and bone. Lele, L. L. (1962) “A simple method for production of trackless focal lesions with focused ultrasound: physical factors,” J. Physiol 160:494-512; Fry et al., (1978) “Tumor irradiation with intense ultrasound”, Ultrasound Med. Biol. 4:337-341. Using an array of ultrasound transducers with high precision of heat deposition, focused ultrasound can be delivered at high intensity to a defined very small area of deep tissue. Focus of the ultrasound is achieved by the shape of the transducer (spherical, parabolical) and/or by combining several different transducer elements and combining their ultrasound waves with individually adjusted phases in order to provide a focal spot. The principles of ultrasound can be found in, for example, Bushberg J. T. et al., The Essential Physics Of Medical Imaging, Williams and Wilkins, Baltimore, 1994, pp. 367-526.

In general, published studies have either sought to use ultrasound to deliberately burn tissue, or to image tissues without significantly raising their temperature. See, e.g., McAllister et al. (1994) Teratology 51:191; Cline et al. (1992) “MR-guided focused ultrasound surgery” J. Comp. Asst. Tomog. 16:956-965. Angles et al. (1991) Teratology 42:285 reported that ultrasound can activate heat shock genes in the absence of any detectable rise in temperature. In U.S. Pat. No. 5,447,858, a soybean hop promoter was recombined with a heterologous gene, introduced into plant cells, and the hap promoter was activated using the “heat of day” (column 11, lines 15-25) or incubation at, for example, 42.5° C. (column 11, line 37). High intensity focused ultrasound has been used to ablate tumors in animal models (Lele (1962), J. Physiol. 160:494-512; Fry et al. (1978), Ultrasound Med. Biol. 4:337341) and is a proposed surgical technique for treating liver tumors (ter Harr et al. (1991), Phys. Med. Biol. 36:1495-1501; ter Harr et al. (1991), Min. Invas. Ther. 1:13-19).

In contrast, the present invention sets out to deliberately heat tissue within a target volume, but in a finely controlled fashion within a defined range of temperatures. In the past, several factors have limited the use of ultrasound to locally heat tissue: 1) the inability to precisely pinpoint the exact location of heat deposition due to interference near air/water, water/bone, and fat/water boundaries, 2) the inability to precisely quantify temperature elevation, and 3) the inability to simultaneously visualize the target tissue and surrounding tissues to monitor extent and effects of ultrasound heating. One possibility is to use a combination of focused ultrasound and magnetic resonance imaging (MRI). Cline et al. (1994) Magn. Reson. Med. 31:628636, Cline et al. (1995) J. Comp. Asst. Tom. 16:956-965, De Poorter (1995) Magn. Reson. Ned. 33:74-81.

It should be noted that the heat shock promoter may be activated by phenomena other than ultrasound that can raise body temperature (e.g., fever, hot shower, stress). Thus, it is appropriate to stringently control these variables (closely monitor a patient's temperature, avoid hot showers, avoid stress-producing environments) during the duration of treatment. Another approach is to limit the duration of the gene therapy.

In addition, heat can activate endogenous,heat shock genes under the control of endogenous hop promoters. The interaction between the processes will be studied.

Further, as noted above, the invention also includes the use of other non-heat inducible stress promoters. These promoters are inducible, e.g. by application of chemicals or heavy metals.

Imaging of Temperature

An element of the noninvasive use of focused ultrasound is that one needs to assure that 1) the heated area corresponds with the target tissue, and 2) the temperature elevation corresponds with the target temperature. The first problem requires anatomical visualization, and the second visualization of temperature distribution.

Whereas ultrasound can in principle be used for both purposes, its precision is considered inadequate for this purpose.

Cline et al., (1992) J. Comp. Asst. Tomog. 16:956-963, described the combination of Magnetic Resonance Imaging (MRI) and focused ultrasound, in which MRI is used to visualize and map the target area and to visualize and map the temperature distribution. The principles of MRI can be found in for example Stark D. D. and Bradley, W. G., Magnatic Resonance Imaging, Mosby Year Book, 1992, pp. 1-521.

Imaging of temperature can be accomplished by MRI in three ways: 1) using the spin-lattice (T1) relaxation dependence on temperature; 2) using the diffusion constant dependence of water on temperature; and 3) using the Larmor-precession frequency dependence of water protons on temperature. It is increasingly clear that the third method is the preferred method since it is rather independent of most intra- and extracellular processes, and it can be measured very rapidly in an imaging method. Cline et al. (1994), “MR temperature mapping of focused ultrasound surgery,” Magn. Reson. Med. 31:628-636; De Poorter et al. (1995), “Noninvasive MRI thermometry with the proton resonance frequency (PRF) method: In vivo results in human muscle”, Magn. Reson. Med. 33:74-819; Hall et al. (1985), “Mapping of pH and temperature distribution using chemicalahift-resolved tomography,” J Magn. Reson. 65:501-505 (J. de Zwart et al. (1996), J. Magn. Reson. Series B, 112:86-90 and references therein).

Proton resonance frequency (PRF) depends on temperature. PRF information is obtained from the phase shift in gradient echo images. MRI Thermometry based on PRF shows little if any dependence on intra- and extracellular composition. Imaging speed is essential for two reasons: avoiding motion artifacts, and limiting effects of thermal conduction on quantitation of temperature increase. In order to minimize total imaging time, the time between successive excitations (repetition time, “TR”) should be short. However, echo time (“TE”) should be long to allow phase accumulation. Using this technology, it is feasible to acquire 3D images within 5 seconds with a spatial resolution of about 3-4 mm, and a temperature accuracy of about 2 degrees C. In the preferred embodiment, a MRI guided focused ultrasound is used as is described in Cline et al., (1995) Magn. Reson. Imaging, 194:731-737. In future embodiments, the single ultrasound transducer under mechanical control described by Cline et al. will preferably be replaced with an array of transducers under electronic control to steer the focus electronically as described by Fan, X. and Hynynen, (1995) Med. Phys. 22(3):297-306.

### EXAMPLES

The following examples are offered solely for the purposes of illustration, and are intended neither to limit nor to define the invention.

### EXAMPLE 1

### Preparation of a Vector Containing a hsp Promoter Operably Linked to a E1b Gene

Vectors derived from adenovirus serotype 5 are used in this example. S. L. Brody and R. G- Crystal (1994), “Adenovirus-Mediated In Vivo Gene Transfer,” Ann. N.Y. Acad. Sci. 716:90-101.

The human hsp-70B promoter is used, as it is strictly heat regulated and can promote a several thousand fold increase in expression upon induction (M. Dreano et al. (1986), “High level heat-regulated synthesis of proteins in eukaryotic cells,” Gene 49:1).

The vector is constructed by inserting the human hsp70 promoter in place of the endogenous E1b promoter in the adenovirus type 5 genome. The E. coli LacZ gene is optionally included in the vector as a reporter gene. Its product, β-galactosidase, can be easily detected and quantified by its specific substrate. An SV40 polyadenylation sequence is optionally used together with a inverse terminal repeat (ITR) as an encapsidation signal and enhancer. Construction of the vector is accomplished using a plasmid containing the cassette and the adenovirus type 5 sequences used for homologous recombination with the E1- or E3 adenovirus genomic DNA.

The modified adenovirus is grown in 293 cells. Viral vectors are produced in titers of up to 10¹² plaque forming units per mL.

### EXAMPLE 2

### Preparation of a Vector Containing a hsp Promoter Operably Linked to a E1a Gene

Vectors derived from adenovirus serotype 5 are used in this example. S. L. Brody and R. G- Crystal (1994), “Adenovirus-Mediated In Vivo Gene Transfer,” Ann. N.Y. Acad. Sci. 716:90-101.

The human hsp-70B promoter is used, as it is strictly heat regulated and can promote a several thousand fold increase in expression upon induction (M. Dreano et al. (1986), “High level heat-regulated synthesis of proteins in eukaryotic cells,” Gene 49:1).

The vector is constructed by inserting the human hsp70 promoter in place of the endogenous E1 a promoter in the adenovirus type 5 genome. The E. coli LacZ gene is optionally included in the vector as a reporter gene. Its product, β-galactosidase, can be easily detected and quantified by its specific substrate. An SV40 polyadenylation is optionally used together with a inverse terminal repeat (ITR) as an encapsidation signal and enhancer. Construction of the vector is accomplished using a plasmid containing the cassette and the adenovirus type 5 sequences used for homologous recombination with the E1- or E3 adenovirus genomic DNA.

The modified adenovirus is grown in 293 cells, a transformed human 12 embryonic kidney call line. Viral vectors are produced in titers of up to 10¹² plaque forming units per mL.

### EXAMPLE 3

### Administration of the Vector to a Patient

The vectors of Examples 1 or 2 are administered systemically to a patient that has been diagnosed as having a mammary adenocarcinoma. Preferably, 1 μg to 100 μg vector DNA are injected in 0.1-2 mls of a saline solution directly into the tumor. Alternatively, approximately 10 μg to 1 mg vector DNA are intravenously injected in 1-5 mls of a saline solution.

The presence and/or absence of the vector is determined by obtaining a biopsy of the cancerous tissue and demonstrating the presence of the gene or gene product by well known Northern, Southern or Western blotting techniques, or by detecting the activity of the optional reporter LacZ gene. As replication of the vector is regulated by hsp70 promoter, the vector will not be appreciably detected unless stress inducible, e.g. elevated temperature, conditions are present.

### EXAMPLE 4

### Focused Ultrasound Heating

A patient is placed on a special bed (e.g., General Electric Co., Milwaukee, Wis., as described in Cline et al. 1994 and 1995, supra) and moved into the magnet of a magnetic resonance imaging (MRI) instrument (e.g., 1.5T Nat Imaging system by Signa, GE Medical Systems, Milwaukee, Wis.). The MRI instrument is equipped with a focused ultrasound (FUS) device (Specialty Engineering Associates, Milpitas, Calif.) under computer control. Specifically, the FUS device can be incorporated in the bed of the MRI in such a way that the transducer can be freely moved under the patient with motional freedom in the three principal directions to allow the focus to be placed anywhere in the human body. Alternatively, the focus can be adjusted electronically by using a more complicated FUS transducer, a so-called phased array FUS transducer, in fact a combination of multiple transducers that can be controlled individually by electronic means thus allowing to move the focus. Acoustic contact between the focus and the FUS transducer is assured using appropriate water, gel, or other means giving an uninterrupted acoustic path from transducer to focus. A Sparc 10 (Sun Microsystems, Mountain View, Calif.) workstation interfaced to the motor controls, the FUS pulse generator and the MR imaging system is used to program, plan, monitor and control therapy. Cline et al., supra, and Zwart et al., supra.

The area of the target is immobilized by gentle straps to the bed. (Note that the more accelerated the procedure, the less the need for immobilization; with very accelerated procedures immobilization is unnecessary.)

Highly detailed MRI images are obtained with a suitable contrast to determine accurately the computer coordinates of the target (e.g. tumor, or ischemic area) as per standard MRI procedures. Based on i) coordinates of the target, ii) estimates of ultrasound attenuation, iii) acoustic impedance transitions in the ultrasound paths, the focus, power and exposure time of the FUS device are targeted to give an increase in temperature of three degrees Celsius in approximately 10 seconds at the target.

The FUS device is switched on for 10 seconds. Immediately following the FUS exposure, a rapid MRI temperature image is taken as per the procedure outlined in J. de Zwart et al. (1996), J. Magn. Reson. Series B, 112:86-90 and references therein). An evaluation is made as to the following criteria: i) Does the heated spot correspond with the target (comparison of anatomical MRI and temperature MgtI), and ii) Is the temperature elevation indeed 3 degrees Celsius (quantification of temperature, see J. de Zwart et al. under 5)? If not, the FUS target is moved in the first case, and the power is adjusted in the second case. The trial heating is repeated until location and power correspond with the target. Note that, since hsp-70B promoter activity is linearly proportional with the duration, gene expression in this adjustment procedure is limited because of its short duration.

Once power and focus have been adjusted, the therapeutic dose of the ultrasound is delivered. For the hsp-70B promoter, an elevation by 3 degrees for 15 minutes gives rise to very large expression of the gene under hsp-70H control. Therefore, the initial exposure is 15 minutes. It can be increased or decreased at the discretion of the attending physician, taking into consideration the severity of the condition treated, the condition (age, health) of the patient, and the size and location of the target area.

The patient is then removed from the MRI. Evaluation of therapy is performed by clinical examination and regular follow-up of detailed anatomical NRI to evaluate tumor shrinkage.

What is claimed is:

1. A modified vector, the replication, immunogenicity and/or pathogenicity of which is not normally under the regulatory control of a stress protein regulatory element, and wherein said vector is modified by the addition of at least one stress protein regulatory element thereby resulting in a modified vector wherein the replication, immunogenicity and/or pathogenicity of which is modulated by said at least one stress protein regulatory element.

2. The modified vector of claim 1, which is selected from the group consisting of a virus, bacterium, yeast, protozoan and a plasmid.

3. The modified vector of claim 1, which is a virus.

4. The modified vector of claim 3, wherein said virus is selected from the group consisting of adenovirus, adeno-associated virus, lentivirus, immunodeficiency virus, herpes virus, baculovirus, cytomegalovirus, paillomavirus, RSV, rhodovirus, vaccinia virus, simian immunodeficiancy virus, hepatis, poxvirus, picornavirus, rhinovirus, and feline leukemia virus.

5. The modified vector of claim 4, which is an adenovirus.

6. The modified adenovirus of claim 5, wherein said heat shock regulatory element controls expression of at least one gene selected from the group consisting of E1a, E1b, E2, E3 and E4.

7. The modified vector of claim 1, which is a plasmid.

8. The modified plasmid of claim 7, which comprises an autonomous replication sequence (ARS) operably linked to a stress protein regulatory element.

9. The modified vector of claim 1, which is a bacterium.

10. The modified bacterium of claim 9, which is selected from a genus selected from the group consisting of Escherichia, Bacillus, Streptococcus, Staphylococcus, Vibrio and Haemophilus.

11. The modified vector of claim 1, wherein said stress protein is selected from the group consisting of CAT5, CPH1, C4P2, DDR2, RPR2, HSC82, HSP47, HSP70.1, HSP90, HSP104, HSP12, HSP150, HSP26, HSP42, HSP60, HSP70, HSP70B, HSP106, HSP78, HSP82, KAR2, MDJ1, S1W1, SUD2, SSA1, SSA2, SSA3, SSB1, SSE1, SSB2, SSC1, SSE2, ST11 , T1P1, TPS2, UB14, 4DT1, ubiquitin, crystalline, rapamycin, P-glycoprotein, metallothionein 1, metallothionein 2, metallothionein 1A and metallothionein 1G.

12. The modified vector of claim 1, wherein said stress protein is a heat shock protein selected form the group consisting of HSP12, HSP26, HSP42, HSP47, HSP70, HSP70B, HSP70.1, HSP60, HSP78, HSP82, HSP90, HSP104 and HSP150.

13. The modified vector of claim 12, wherein said stress protein is that of a yeast, insect, mammalian, or amphibian.

14. The modified vector of claim 12, wherein said stress protein is that of a Drosophila, monkey, frog, mouse, or human.

15. The modified vector of claim 12, wherein said stress protein is HSP70 or HSP70B.

16. A method of gene therapy which comprises administration of a vector according to claim 1 to a subject in need of such treatment;
exposing said subject to stress conditions at a particular site in order to induce the expression of a gene contained on the vector which affects replication, pathogenicity and/or immunogenicity.

17. The method of claim 16, wherein the vector is a virus.

18. The method of claim 17, wherein said virus is selected from the group consisting of vaccinia virus, adenovirus, herpes virus, adeno-associated virus, hepatitis virus, lentivirus and cytomegalovirus.

19. The method of claim 18, wherein said virus is an adenovirus.

20. The method of claim 19, wherein said adenovirus contains a replication gene selected from E1a and/or E1b regulated by a heat shock promoter.

21. The method of claim 20, wherein said adenovirus further comprises a gene encoding a cytotoxic agent, therapeutic agent, anti-proliferation agent, differentiation inducing agent and anti-angiogenic agent.

22. The method of claim 20, wherein said heat shock promoter is HSP70 or HSP70B.

23. The method of claim 16, which is used to treat cancer.

24. The method of claim 16, which is used to treat cystic fibrosis.

25. The method of claim 16, which is used to treat an autoimmune disorder

26. A method of effecting specific cell or tissue ablation comprising administrating a modified vector according to claim 1 to a subject in need of such cell or tissue ablation;
exposing an area of said subject in need of cell or tissue ablation to stress conditions that induce the replication, immunogenicity and/or pathogenicity of said vector thereby resulting in the selective ablation of cells or tissues at target site.

27. The method of claim 26, wherein the vector is a virus.

28. The method of claim 27, wherein said virus is selected from the group consisting of a lentivirus, poxvirus, adenovirus, adeno-associated virus, herpes virus, hepatitis virus and cytomegalovirus.

29. The method of claim 27, wherein said cell or tissue ablation is used for the treatment of cancer.

30. The method of claim 28, wherein said virus is an adenovirus.

31. The method of claim 30, wherein said adenovirus contains an E1a or E1b gene and/or the regulatory control of a stress-inducible promoter.

32. The method of claim 31, wherein said promoter is HSP70 or HSP70B.

33. The method of claim 27, wherein the vector is a bacterium or yeast.

34. The method of claim 27, wherein the vector is a plasmid.

35. The method of claim 33, wherein said bacterium or yeast comprises a gene that affects proliferation under the regulatory control of a stress-inducible promoter.

36. A method of vaccinating a host against a target antigen comprising:
(i) administering a modified vector according to claim 1 which comprises a gene encoding a target antigen under the control of a stress-inhibitor regulating sequence to a subject to be immunized against the target antigen;
(ii) inducing the expression of said gene at a target site by exposing said site to stress conditions; and
(iii) maintaining said stress conditions for a time sufficient to allow said host to elicit an immune response to said target antigen.

37. The method of claim 36, wherein said target antigen is an immunodominant epitope of a virus, bacterium, protozoan or yeast.

38. The method of claim 37, wherein said target antigen is a viral capsid protein or epitope thereof.

39. The method of claim 36, wherein said subject is “boosted” with additional antigen after the initial induction by again exposing the subject to stress conditions.

40. A method of identifying the function of a target gene comprising the following steps:
(i) administering to an animal a modified vector according to claim 1 further containing a gene, the function of which is to be assayed;
(ii) assessing the phenotype change that the expression of said gene causes upon induction of expression by exposure of the subject to stress-induction conditions.
